(19) United States
(12) Patent Application Publication
(10) Pub. No.: US 2024/0151679 A1
(43) Pub. Date: May 9, 2024

(54) ELECTROCHEMICAL SENSOR FOR PHENOLIC ANALYTES

(71) Applicant: EYE3CONCEPTS INC., Oakville (CA)

(72) Inventors: Greter Amelia ORTEGA RODRIGUEZ, Hamilton (CA); Satish Kumar TUTEJA, Hamilton (CA); Syed Rahin AHMED, Hamilton (CA); Seshasai SRINIVASAN, Brampton (CA); Amin Reza RAJABZADEH, Burlington (CA)

(21) Appl. No.: 18/277,844

(22) PCT Filed: Feb. 18, 2022
(86) PCT No.: PCT/CA2022/050234

§ 371 Date: Aug. 18, 2023

Related U.S. Application Data
(60) Provisional application No. 63/151,339, filed on Feb. 19, 2021.

(51) Int. Cl. G01N 27/327; G01N 27/48; G01N 33/94

(52) U.S. Cl. CPC G01N 27/3277; G01N 27/48; G01N 33/948

(57) ABSTRACT

The present disclosure concerns a sensor for detecting a sample analyte having an oxidizable phenolic group suspected to be present in a sample. The sensor comprises a sensing electrode, a plurality of sensor analytes associated with the sensing electrode, optionally a baseline electrode, and a sample receiving region in fluid communication with the sensing electrode. The sensor analyte, before its association with the sensing electrode, is the same chemical species as the sample analyte.

### CROSS-REFERENCE TO A RELATED APPLICATION

The present application claims priority from U.S. provisional application Ser. No. 63/151339 filed Feb. 19, 2021 which is incorporated herein by reference in its entirety.

### TECHNICAL FIELD

The present disclosure generally relates to the field of electrochemical sensors, particularly sensors that detect analytes having an oxidizable phenol group.

### BACKGROUND

Electrochemical sensors are useful for the detection of various analytes. However, their use has been limited in detecting minute amounts of analytes in complex biological samples, such as bodily fluids. Bodily fluids, such as saliva, for example, comprise many electroactive agents which can significantly interfere with the measurement signal associated with the target analyte. Existing detection methods can rely on pre-treating the sample to enrich the target analyte (by using capturing agents like receptors, antibodies, and aptamers for example) and/or removing the electroactive agents from the sample. Existing detection methods can also rely on an incubation step to allow prolonged contact between the sample and the electrochemical sensor. The use of capturing agents or a prolonged incubation time is not compatible with target analytes having a tendency to easily oxidize or with rapid testing (e.g., under 5 minutes). Existing methods can also rely on the use of a buffer during the detection step.

(—)-trans-Δ⁹-tetrahydrocannabinol (THC) is the most psychoactive component of Cannabis sativa L. Many countries have established policies or laws permitting the legal consumption of cannabis for medicinal, religious, and/or recreational purposes. On the other hand, where cannabis has not been legalized, it is also the most widely used illicit drug. Therefore, the wide use, growing availability, and the psychoactive effects are reasonable motives for the development of detection methods for THC. More specifically, a rapid, portable oral fluid (OF) testing device for THC detection would allow for roadside screening to identify drivers under the possible influence of this drug among other potential applications.

There is therefore considerable utility in developing a sensor that is able to detect minute amounts of analytes in a complex sample such as, for example, a biological sample without the need for capturing agents or long incubation times.

### SUMMARY

The present disclosure comprises a sensor for detecting a sample analyte having an oxidizable phenolic group using a sensor analyte being and/or derived from the sample analyte.

According to a first aspect, the present disclosure provides a sensor for detecting a sample analyte having an oxidizable phenolic group suspected to be present in a sample. The sensor comprises a sensing electrode, a plurality of sensor analytes associated with the sensing electrode, and a sample receiving region in fluid communication with the sensing electrode. The plurality of sensor analytes facilitate, in the presence of an electric potential, (i) oxidation of the oxidizable phenolic group of the sample analyte to obtain an oxidized sample analyte, and (ii) association of a portion of the oxidized sample analyte with the sensing electrode. The plurality of sensor analytes comprise at least one of the following chemical species: a first chemical species corresponding to the sample analyte having the oxidizable phenolic group, a second chemical species corresponding to the sample analyte oxidized at the oxidizable phenolic group, and a third chemical species corresponding to a dimer, an oligomer or a polymer of the sample analyte oxidized at the oxidizable phenolic group or a combination thereof. In an embodiment, the sensor further comprises a baseline electrode, wherein the baseline electrode is a bare working electrode. In another embodiment, the sensor further comprises at least one substrate receiving the sensing electrode and/or the baseline electrode. In yet another embodiment, the plurality of sensor analytes comprise a cannabinoid and/or is derived, at least in part, from oxidation, dimerization, oligomerization or polymerization of the cannabinoid. In a specific embodiment, the cannabinoid is Δ⁹-tetrahydrocannabinol (THC). In a further embodiment, the sensing electrode comprises a carbon-based material, a nanomaterial, a metal-based material, or a combination thereof. In another embodiment, the sample comprises a bodily fluid or a component of the bodily fluid. In a specific embodiment, the sample comprises saliva or a component of saliva. In another embodiment, the sensor is for detecting the sample analyte with a voltammetric technique, such as, for example, square wave voltammetry, cyclic voltammetry, linear sweep voltammetry, or differential pulse voltammetry.

According to a second aspect, the present disclosure provides a packaging comprising the sensor described herein in an inert atmosphere or vacuum-sealed.

According to a third aspect, the present disclosure provides a kit comprising the sensor described herein and a detection device.

According to a fourth aspect, the present disclosure provides a process of fabricating a sensor for detecting a sample analyte having an oxidizable phenolic group suspected of being present in a sample. The process comprises (i) contacting a sensing electrode with a plurality of sensor analytes, wherein the plurality of sensor analytes comprises a first chemical species corresponding to the sample analyte having the oxidizable phenolic group; and (ii) associating the plurality of the sensor analytes with the sensing electrode. The plurality of sensor analytes associated with the sensing electrode facilitate, in the presence of an electric potential, (i) oxidation of the oxidizable phenolic group of the sample analyte to obtain an oxidized sample analyte, and (ii) association of a portion of the oxidized sample analyte with the sensing electrode. The plurality of sensor analytes comprise at least one of the following chemical species: the first chemical species corresponding to the sample analyte having the oxidizable phenolic group, a second chemical species corresponding to the sample analyte oxidized at the oxidizable phenolic group, a third chemical species corresponding to a dimer, an oligomer or a polymer of the sample analyte oxidized at the oxidizable phenolic group or a combination thereof. In an embodiment, the plurality of sensor analytes are provided in a solution. In another embodiment, the solution comprises an organic solvent, such as, for example, methanol. In still another embodiment, the process further comprises, prior to step (i), diluting the plurality of sensor analytes in an oxidizable form in the solution. In yet another embodiment, the process further comprises washing and/or polishing the working electrode. In an embodiment, step (ii) comprises applying at least one potential to the sensing electrode to associate the plurality of the sensor analytes. In another embodiment, step (ii) comprises electrodepositing the plurality of sensor analytes on the sensing electrode. In an embodiment, the first chemical species of the plurality of sensor analytes is a cannabinoid, such as, for example, Δ⁹-tetrahydrocannabinol. In still another embodiment, the process further comprises, after step (ii), determining an electric current component resulting from the first chemical species of the plurality of sensor analytes associated with the sensing electrode in the presence of the electric potential.

According to a fifth aspect, the present disclosure provides a sensor obtained by the process described herein.

According to a sixth aspect, the present disclosure provides a method of detecting a sample analyte having an oxidizable phenolic group suspected to be present in a sample. The method comprises receiving the sample on a sensor described herein, applying the electric potential to the sensing electrode to oxidize the sample analyte and obtain an oxidized sample analyte, and to associate a portion of the oxidized sample analyte with a surface of the sensing electrode, obtaining a measured current from the sensing electrode while the at least one electric potential is applied, comparing the measured current to a baseline current to determine a difference, the baseline current resulting from interfering electroactive agents in the sample; and detecting a presence of the sample analyte when the difference exceeds a detection threshold. In an embodiment, the method further comprises receiving the sample on a sample receiving region of a baseline electrode, applying the electric potential to the baseline electrode and measuring the baseline current while the electric potential is applied, wherein the baseline electrode is a bare working electrode. In yet another embodiment, the difference is proportional to a concentration of the sample analyte in the sample. In a further embodiment, the method further comprises quantifying a concentration of the analyte as a function of the difference. In yet another embodiment, the electric potential is applied and the measured current is obtained using a voltammetric technique such as, for example, square wave voltammetry, cyclic voltammetry, linear sweep voltammetry, or differential pulse voltammetry. In an embodiment, the sample analyte is a cannabinoid such as, for example, Δ⁹-tetrahydrocannabinol (THC). In an embodiment, applying the electric potential comprises applying a plurality of electric potentials in a range of between 0 to 1 V to the sensing electrode. In a further embodiment, the sample comprises a body fluid or a component of the bodily fluid. In still another embodiment, the sample comprises saliva or a component of saliva. In yet another embodiment, the method has a limit of detection of the sample analyte being equal to or less than about 100 ng/mL. In yet a further embodiment, the method further comprises, before receiving the sample, providing and/or processing the sample. In an embodiment, processing the sample comprises filtering the sample. In still another embodiment, comparing the measured current to the baseline current comprises taking into account a calibration current resulting from a contribution of the sensor analytes on the sensing electrode.

Many further features and combinations thereof concerning the present improvements will appear to those skilled in the art following a reading of the instant disclosure.

### DESCRIPTION OF THE DRAWINGS

FIGS. 1A to 1G are top views of example embodiments of a a sensor for detecting a sample analyte having an oxidizable phenolic group.

FIG. 2 is a cross-sectional view of an embodiment of the sensor illustrating fabrication steps.

FIG. 3 is a block diagram of a detection system for detecting the sample analyte.

FIG. 4 is a schematic diagram illustrating the detection principles of the sensor.

FIG. 5 is a flow chart of an example method for detecting the presence of a sample analyte in a sample.

FIGS. 6A and 6B show the fabrication and calibration of a modified working electrode according to an embodiment of the present disclosure. FIG. 6A shows a schematic representation of the deposition of THC molecules (THCᵢ) on the modified working electrode, and the application of a current (including a reaction scheme). FIG. 6B shows a graph of the corresponding signal (current (μA)) for the deposited THC molecules in PBS in function of electric potential (V).

FIGS. 7A and 7B show scanning electron microscopy (SEM) images. FIG. 7A show representative SEM images of a bare working electrode (e.g., an embodiment of a baseline electrode) at two magnifications (left panel=×500, scale bar 10 μm; right pane=×100,000 m, scale bar 100 nm). FIG. 7B are a representative SEM image of a modified working electrode according to an embodiment of the present disclosure with THC molecules on its surface (left panel=×500, scale bar 10 μm; right panel=×100 000 m, scale bar 100 nm).

FIGS. 8A to 8I show different cyclic voltammetry (CV) graphs of current (μA) vs electric potential (V) at different scan rates from 0.025 to 0.2 V/s. FIG. 8A shows the CV graph of K₄[FeCN₆] (FeCN) 0.1 mM in KCl 0.1 M. FIG. 8B shows the CV graph of simulated saliva (SS) on a bare working electrode. FIG. 8C shows the CV graph of THC (1 000 ng/mL) dispersed in PBS pH 7.4 on a bare working electrode. FIG. 8D shows the CV graph of THC (1 000 ng/mL) dispersed in SS on a bare working electrode. FIG. 8E shows the CV graph of K_{4[}FeCN₆] (FeCN) 0.1 mM in KCl 0.1 M on a modified working electrode. FIG. 8F shows the CV graph of K₄[FeCN₆] (FeCN) 0.1 mM in SS on a modified working electrode. FIG. 8G shows the CV graph of THC (1 000 ng/mL) dispersed in PBS pH 7.4 on the modified working electrode electrode FIG. 8H shows the CV graph of THC (1000 ng/mL) dispersed in SS on on the modified working electrode. FIG. 8I shows the CV graph at different scan rates from 0.025 to 0.2 V/s and 10 cycles of CV at 0.1 V/s (inside) of the modified working electrode in PBS as the electrolyte.

FIGS. 9A to 9D show the electrochemical properties of different electrodes. FIG. 9A shows a plot of the current of the anodic peak (I) vs the square root of the scan rate (v^{1/2}) for FeCN systems (Bare_working_electrode_FeCN_KCl (▪), Bare_working_electrode_FeCN_SS (▴), (Modified_working_electrode_FeCN_KCl (▾), Modified_working_electrode_FeCN_SS ( [symbol] )). FIG. 9B shows a plot of the current of the anodic peak (I) vs of the scan rate (v) for THC systems systems (Bare_electrode_THC_PBS (▪), Modified_working_electrode_PBS (▴), (Modified_working_electrode_THC_PBS (▾), Modified_working_electrode_THC_SS ( [symbol] )). FIG. 9C shows a plot of the variation of the oxidation potential of THC (E) vs the scan rate (v) (Bare_electrode_THC_PBS (▪), Modified_working_electrode_PBS (▾), (Modified_working_electrode_THC_PBS (▴), Modified_working_electrode_THC_SS ( [symbol] )). FIG. 9D shows a plot of the Laviron's equation (Bare_working_electrode_THC_PBS (▪), Modified_working_electrode_PBS (▴), (Modified_working_electrode_THC_PBS (▴), Modified_working_electrode_THC_SS ( [symbol] )).

FIGS. 10A and 10B show the electrochemical properties of electrodes in the presence of different concentrations of THC. FIG. 10A shows a graph of a square wave corresponding to different concentrations of THC in PBS and using bare working electrodes (Bare working electrode-THC in PBS 10,000 ng/mL ( [symbol] ), Bare working electrode-THC in PBS 100 ng/mL (X), Bare working electrode-THC in PBS 10 ng/mL (I)). FIG. 10B shows a graph comparing the SWV signals of a solution of THC 10 ng/mL using a bare working and a modified working electrode according to an embodiment of the present disclosure (Modified_working_electrode-THC in PBS 10 ng/mL ( [symbol] ), Modified_working_electrode-PBS (THC 0 ng/mL) ( [symbol] ), Bare_working_electrode-THC in PBS 10 ng/mL (I)).

FIGS. 11A to 11G show results where the samples were THC 10 ng/mL in PBS, and PBS as control. FIG. 11A shows a graph of the current (μA) vs the electric potential (V) for SWV techniques (modified working electrode-THC in PBS (10 ng/mL): SWV parameters determined herein ( [symbol] ) and SWV Compton parameters ( [symbol] ) modified working electrode in PBS (0 ng/mL THC): SWV parameters determined herein ( [symbol] ) and SWV Compton parameters ( [symbol] )). FIG. 11B shows a graph of the current (μA) vs the electric potential (V) demonstrating the effect of ambient conditions on an embodiment of the sensor electrode (bare working electrodes_THCᵢ ( [symbol] ), modified working electrode—PBS 0 h ( [symbol] ), modified working electrode PBS 24 h ( [symbol] )). FIG. 11C shows a graph of the current (μA) vs the electric potential (V) for different incubation times of the samples on the modified working electrode (modified working_electrodes-THC in PBS (10 ng/mL) at 0 min incubation ( [symbol] ) and at 5 min incubation ( [symbol] ). FIG. 11D shows a graph of the current (μA) vs the electric potential (V) for SWV with different pH of the media (modified working electrode in PBS with 10 ng/mL THC at pH 5.6 ( [symbol] ), at pH 6.6 ( [symbol] ), at pH 7.4, ( [symbol] ), and at pH 8 (. [symbol] ) and modified working electrode in PBS with 0 ng/mL THC at pH 5.6 ( [symbol] ), at pH 6.6 ( [symbol] ), at pH 7.4 ( [symbol] ), and at pH 8 (. [symbol] )). FIG. 11E shows a graph of the current (μA) vs the electric potential (V) for different electrochemical voltammetric techniques (modified working electrode in PBS with 10 ng/mL THC using SWV ( [symbol] ), LSV ( [symbol] ), and DPV ( [symbol] ), and modified working electrode in PBS with 0 ng/mL THC using SWV ( [symbol] ), LSV, ( [symbol] ), and DPV ( [symbol] )), FIG. 11F shows a line plot of electric potential (V) as a function of PBS pH and a bar graph of the difference in current intensity (μA) between THC 10 ng/mL and THC 0 ng/mL also as a function of PBS pH. FIG. 11G shows a line plot of the electride potential (V) of the THC oxidation peaks for different voltametric techniques (SWV, LSV, DPV, and CV) and a bar graph of the difference in current intensity (μA) between THC 10 ng/mL and THC 0 ng/mL vs the different voltametric techniques used (SWV, LSV, DPV, and CV).

FIGS. 12A to 12D show the properties of an embodiment of a modified working electrode. FIG. 12A shows a schematic representation of the deposition of THC on the modified working electrode followed by interrogation with SWV in PBS pH 7.4 or simulated saliva (SS). FIG. 12B shows a graph of the current (μA) vs the electric potential (V) for SWV of the modified working electrode during the deposition of THCᵢ (A) (10 different modified working electrodes each a solid line) and then in PBS pH 7.4 (B) (10 different modified working electrodes each a dotted line). FIG. 12C shows a graph of the modified working electrode current (μA) vs THCᵢ current (μA) to determine the mathematical correlation between ITHCᵢ and I₀ of the modified working electrode in PBS (each ▪ represents a different modified working electrode and the line is a polynomial fit (R=0.94) I₀=−0.2[±0.1]+0.7[±0.2]ITHCᵢ−0.15[±0.10]I² THCᵢ). FIG. 12D corresponds to the graph with simulated saliva (each ▪ represents a different modified working electrode and the line is a polynomial fit (R=0.97) I₀=−0.12[±0.02]+0.14[±0.02] ITHCᵢ).

FIGS. 13A to 13C show the electrochemical properties of electrodes in the presence of different concentrations of THC. FIG. 13A shows a graph of current (μA) vs electric potential (V) for SWV of THC in PBS at different concentrations using modified working electrodes according to one embodiment of the present disclosure (THC 0 ng/mL (●), THC 2 ng/mL ( [symbol] ), THC 5 ng/mL ( [symbol] ) and THC 10 ng/mL ( [symbol] )). FIG. 13B shows a bar graph of current intensities of the THCᵢ for each electrode and, on their right side, the intensity of the sample interrogating with the same electrode (THCᵢ ( [symbol] ), THC 10 ng/mL ( [symbol] ), THC 5 ng/mL ( [symbol] ), THC 2 ng/mL ( [symbol] ), and THC 0 ng/mL ( [symbol] )). FIG. 13C shows two bar graphs of the current intensity values (μA) vs the THC concentration (ng/mL) before (on the left) and after (on the right) the correction with I₀.

FIGS. 14A to 14D shows the electrochemical properties of electrodes with different concentrations of THC in simulated saliva. FIG. 14A shows a graph of current (μA) vs electric potential (V) for SWV of THC in PBS for different THC concentration on the modified working electrodes (7 electrodes per concentration) (1000 ng/mL ( [symbol] ), 100 ng/mL ( [symbol] ), 10 ng/mL ( [symbol] ), 5 ng/mL)( [symbol] ), 2 ng/mL ( [symbol] ), and 0 ng/mL (●)). FIG. 14B shows a graph of the current difference between the current measured with the sample and I₀ (μA) vs THC concentration for the modified working electrode simulated with THC in PBS, the logistic fit (R =0.98) for the calibration curve was I_{mWE}−I₀=A₂+(A₁−A₂)/(1+(x/x₀)^{P}) with A₁=−0.019±0.009, A₂=0.29±0.03, x₀=10±3, p=1.3±0.4), the limit of detection (LD) was determined to be 1.1 ng/mL. The limit of detection was calculated by considering the minimal signal to be positive as the sum of the signal of the blank plus 3 times the standard deviation of the blank for a 95% of the confidence interval. FIG. 14C shows a graph of the current (μA) vs the electric potential (V) for SWV of THC in simulated saliva for different THC concentration on the modified working electrodes (7 electrodes per concentration) (1000 ng/mL ( [symbol] ), 100 ng/mL ( [symbol] ), 10 ng/mL ( [symbol] ), 5 ng/mL ( [symbol] ), 2 ng/mL ( [symbol] ), and 0 ng/mL (●)). FIG. 14D shows a graph of the current difference between the current measured with the sample and I₀ (μA) vs the THC concentration for the modified working electrode simulated with THC in simulated saliva, the logistic fit (R=0.93) for the calibration curve was I_{mWE}−I₀=A₂+(A₁−A₂)/(1+(x/x₀)^{P}) with A₁=−0.003±0.009, A₂=0.12±0.01, x₀=4±3, p=1.5±0.9). The limit of detection was determined to be 1.6 ng/mL, the error bars for 95% of confidence intervals are represented.

FIGS. 15A and 15B show the results of an interference assay. FIG. 15A shows a graph of the current (μA) vs the electric potential (V) with two different current scales on a bare working electrode, PBS (♦), inorganic components (IC) (⋄), biological components (BC) (⋄), organic components (OC) ( [symbol] ), uric acid 0.05 mg/mL (UA) ( [symbol] ), and biological saliva ( ). FIG. 15B shows a graph of the current (μA) vs the electric potential (V) with two different current scales on a modified working electrode, PBS (▴), inorganic components (IC) (⋄), biological components (BC) (∇), organic components (OC) (□), uric acid 0.05 mg /mL (UA) (▪), and biological saliva ( [symbol] ).

FIGS. 16A to FIG. 16D show the results obtained in real saliva. FIG. 16A shows a schematic representation of the detection of THC in biological saliva samples interrogated with bare and modified working electrodes at the same time. The graph in the middle shows the current (μA) vs the electric potential (V) for the modified working electrode stimulated with 10 ng/mL THC in saliva (●) and the bare working electrode stimulated with 10 ng/mL THC in saliva ( [symbol] ). The graph on the right shows the subtraction of both signals (I_{mWE}−I_{b}) vs the electric potential (V), which was proportional to the THC concentration in saliva. FIG. 16B shows a graph of the current (μA) vs the electric potential (V) for SWV of THC in saliva (modified working electrode THC: 0 ng/mL (●), 2 ng/mL (o), 5 ng/mL ( [symbol] ), and 10 ng/mL ( [symbol] ), bare working electrode THC: 0 ng/mL ( [symbol] ), 2 ng/mL ( [symbol] ), 5 ng/mL ( [symbol] ), and 10 ng/mL ( [symbol] )). FIG. 16C further shows subtraction of the current of B (modified working electrode minus bare working electrode) for each concentration of THC (0 ng/mL (●), 2 ng/mL (o), 5 ng/mL ( [symbol] ), and 10 ng/mL ( [symbol] )). FIG. 16D shows the calibration curve of the current different from C vs the concentration of THC, an exponential fit for THC in saliva was obtained (R=0.98). The limit of detection was calculated by considering the minimal signal to be positive as the sum of the signal of the blank plus 3 times the standard deviation of the blank for 95% of the confidence interval. The equation obtained was I_{mWE}−I_{b}=a−b*c^{x} with a=0.27±0.01, b=0.15±0.01, and c=0.75±0.07. The limit of detection was 3.4 ng/mL.

FIGS. 17A to FIG. 17C show the results obtained in real saliva. In all three graphs of current difference vs the THC concentration the initial amount of THC deposited on the modified working electrode was 30 ng. FIG. 17A shows the calibration curve of the current different from C vs the concentration of THC, an exponential fit for THC in saliva was obtained (R=0.98). The limit of detection was calculated by considering the minimal signal to be positive as the sum of the signal of the blank plus 3 times the standard deviation of the blank for 95% of the confidence interval. The equation obtained was I_{mWE}−I_{b}=a−b*c^{x} with a=0.27±0.01, b=0.15±0.01, and c=0.75±0.07. The limit of detection was 3.4 ng/mL. FIG. 17B shows the calibration curve of the current different from C vs the concentration of THC, an exponential fit for THC in saliva adjusted to pH 8, no proper fit was obtained. FIG. 17C shows the calibration curve of the current different from C vs the concentration of THC, an exponential fit for THC in diluted saliva (1:1) was obtained (R=0.94). The equation was I_{mWE}−I_{b}=a−b×with a=0.17±0.01, and b=0.020±0.003. The limit of detection was calculated by considering the minimal signal to be positive as the sum of the signal of the blank plus 3 times the standard deviation of the blank for 95% of the confidence interval.

FIGS. 18A to 18C show the results obtained with different concentrations of THC deposited. FIG. 18A shows a graph of the current (μA) vs the electric potential (V) for SWV during the deposition of different amounts 50 ( [symbol] ), 70 ( [symbol] ), and 100 ( ) ng of THCᵢ and the corresponding signals for the modified working electrode in PBS (50 ng ( [symbol] ), 70 ng ( [symbol] ), and 100 ng ( ) FIG. 18B shows a graph of the current (μA) vs the electric potential (V) for SWV of modified working electrodes (n=5) with the previous deposition of 100 ng of THCᵢ in the presence of PBS ( [symbol] ) and a mix of solvents PBS: methanol (1:1) ( ). FIG. 18C shows a graph on the left of the current (μA) vs the electric potential (V) for SWV of modified working electrodes (n=5) with the previous deposition of 100 ng of THCᵢ for a modified working electrode with saliva: methanol (9:1) (o), and saliva: methanol: PBS (5:1:4) ( ) and for a bare working electrode in the presence of saliva:methanol (9:1) (o) and saliva:methanol:PBS (5:1:4) (+), and the graph on the right side shows the peaks after subtracting the bare working electrode current from the modifeied working electrode signal vs the electric potential (V).

FIGS. 19A to 19D show embodiments of calibration curves. The amount of THC initially deposited on the working electrodes was 100 ng, more than 15 times (I_{mWE}−I_{b}) per concentration, and different saliva samples were tested, the error bars were calculated by considering a 95% level of confidence. FIG. 19A shows a calibration curve (CC) of current intensity difference (I_{mWE}−I_{b}) (μA) vs the concentration of THC (ng/mL) with different saliva dilution ratios Saliva:methanol:PBS (900:100:0 (●), 800:200:0 ( [symbol] ), 800:200:100 (▴), 700:0:300 (▾), 700:50:300 (Δ), 700:100:200 (□), 700:200:100 (♦), and 500:100:400 ( )). FIG. 19B shows the curve fit of the calibration curve for saliva:methanol at 900:100 (R=0.99) the equation of the fit was I_{mWE}−I_{b}=a−b*c^{x} with a=0.943±0.003, b=0.343±0.003, and c=0.733±0.006, and the limit of detection was 1.6 ng/mL. FIG. 19C shows the linear fit of the calibration for saliva:methanol:PBS at 500:100:400 (R=0.95). The equation was I_{mWE}−I_{b}=a−b x with a=0.022±0.006 and b=0.0043±0.0005. The limit of detection was 3.5 ng/mL. FIG. 19D shows the curve fit of the calibration curve for saliva:PBS (700:300) (R=0.99). The equation of the fit was I_{mWE}−I_{b}=a−b*c^{x} with a=0.91±0.008, b=0.54±0.09, c=0.92±0.02. The limit of detection was 2 ng/mL.

FIGS. 20A to 20D show the results obtained in saliva. FIG. 20A shows a graph of the current (μA) vs the electric potential (V) of SWV performances of saliva corresponding to 4 different individuals (individual 1 ( [symbol] ), individual 2 ( [symbol] ), individual 3 ( [symbol] ), and individual 4( [symbol] )). FIG. 20B shows a graph of the current (μA) vs the electric potential (V) of SWV signals obtained after subtracting the SWV voltammograms of modified working electrode minus bare working electrode corresponding to 4 different individuals (individual 1 ( [symbol] ), individual 2 ( [symbol] ), individual 3 ( [symbol] ), and individual 4( [symbol] )) saliva spiked with different concentrations of THC (0 ng/mL, 2ng/mL, 5 ng/mL and 10 ng/mL) at a dilution of saliva: methanol (900:100). FIG. 20C shows a graph of current intensity difference (I_{mWE}−I_{b}) (μA) vs the concentration of THC (ng/mL) for the interpolation of the values obtained from the different samples in the calibration curve in 15B with the dilution saliva: methanol (900:100) (THC in saliva:methanol (900:100) ( [symbol] ), and biological saliva from individuals ( [symbol] )). The limit of detection was 1.6 ng/mL. FIG. 20D shows a bar graph of the concentration of THC (ng/mL) for the four different individuals (I1, I2, I3, and I4) obtained with an ELISA test ( [symbol] ) and the modified working electrode according to an embodiment of the present disclosure ( [symbol] ).

### DETAILED DESCRIPTION

The present disclosure concerns sensors comprising a sensing electrode associated with a plurality of sensor analytes for the detection and optionally the quantification of a sample analyte in a sample potentially comprising interfering electroactive agents (such as, for example, a biological sample). The sensors of the present disclosure do not rely on the use of capturing agents to detect and optionally quantify the sample analyte. The sensors of the present disclosure do not require to be incubated with the sample to provide a measurement when the sample analyte is present in the sample. In some embodiments, the sensors of the present disclosure do not require using a buffer for detecting the sample analyte. In other embodiments, a buffer, such as a dry buffer can be used. Examples of dry buffers include but are not limited to lyophilized (freeze-dried) beads, buffer reagents dried on a membrane, and direct spraying and immobilization of reagents on a surface. In some embodiments, the sensors of the present disclosure can be used as non-invasive or extracorporeal devices for detecting and optionally quantifying a sample analyte in a sample comprising interfering electroactive agents. In some embodiments, the sensors can be used to detect low levels of the sample analyte (e.g., below 100 ng/mL, 50 ng/mL, 25 ng/mL, 10 ng/mL, 5 ng/mL or less). In some embodiments, the sensors can be used to detect ultra-low levels of the sample analyte (e.g., between about 1-10 ng/mL or 1-25 ng/mL) In some embodiments, the sensors can be used to rapidly detect a sample analyte (e.g., between 1 and 10 minutes, or less than 5, 4, 3, 2 or 1 minutes). In some embodiments, the detection using the sensors of the present disclosure can be performed entirely in the field using a handheld or portable device and does not require a controlled setting such as a laboratory. As used herein, the term “portable” can refer to a device that can be carried or moved, such as with little or no effort. The term can also refer to a device that is not permanently affixed to a structure and is of sufficiently low mass and bulk that it may be easily transported in a vehicle, a transportation device or be held in a hand. In some embodiments, the device and/or the sensor can be disposable.

### Sensor Comprising a Sensing Electrode

The sensor of the present disclosure is for the detection of a sample analyte having an oxidizable phenolic group. The sample analyte is suspected to be present in a sample potentially comprising electroactive agents capable of interfering with the detection of the sample analyte. The sample analyte is intended to be oxidized by the sensing electrode of the sensor during detection and thus facilitate its detection.

The sensor of the present disclosure comprises a sensing electrode which is associated or operatively coupled with a plurality of sensor analytes. As used in the context of the present disclosure, the sensing electrode is a working electrode designed to facilitate the oxidation of the sample analyte. As used in the context of the present disclosure, a sensor analyte is a chemical species or a mixture of chemical species which is/are associated (directly or indirectly) with the sensing electrode prior to the detection of the sample analyte.

The association between the sensor analyte and the sensing electrode can be caused by any physical or chemical interaction (or a combination thereof), including, but not limited to ionic interactions, covalent interactions, hydrogen interactions, van der Waals interactions, and/or electrostatic interactions. In some embodiments, the sensor analytes protrude, at least partially, from the surface of the sensing electrode. The sensor analytes can be adsorbed, at least in part, on the surface of the sensing electrode. The sensor analytes can be immobilized, at least in part, on the surface of the sensing electrode. The sensor analytes can be embedded, at least in part, in the sensing electrode.

A portion of the plurality of sensor analytes can be directly associated with the surface of the sensing electrode and/or interact directly with the surface of the sensing electrode. In some embodiments, a portion of the plurality of sensor analytes can be indirectly associated with the surface of the sensing electrode. In such embodiments, the sensor analytes can be associated with one or more sensor analytes which is directly associated with the surface of the sensing electrode. In some specific embodiments, the sensor analytes can be integrated into a dimer, an oligomer, or a polymer of one or more species of the sensor analytes in which at least one monomeric unit is directly associated with the surface of the sensing electrode.

The plurality of sensor analytes on the sensing electrode can comprise one or more (in any proportion) different chemical species. The plurality of sensor analytes can include a first chemical species corresponding to the sample analyte having an oxidizable phenolic group. In such embodiments, the sensor analytes and the sample analyte (prior to detection) correspond to the same chemical species. The plurality of sensor analytes can include a second chemical species corresponding to the sample analyte oxidized at the oxidizable phenolic group. The plurality of sensor analytes can include a third chemical species corresponding to a dimer, an oligomer, or a polymer of the sample analyte oxidized at the oxidizable phenolic group. The plurality of sensor analytes on the sensing electrode can comprise, in any proportion, the first, second, and third chemical species described herein. In a specific embodiment, the plurality of sensor analytes comprises the first and the second chemical species described herein. In a specific embodiment, the plurality of sensor analytes comprises the second and third chemical species described herein. In a specific embodiment, the plurality of sensor analytes comprises the first and the third chemical species described herein. In a specific embodiment, the plurality of sensor analytes comprises the first, the second, and the third chemical species described herein.

The plurality of sensor analytes associated with the sensing electrode facilitates, in the presence of an electric potential, the oxidation of the oxidizable phenolic group of the sample analyte to generate an oxidized sample analyte. A portion of the oxidized sample analyte will be associated with the surface of the sensing electrode. The oxidized sample analytes can be, at least in part, directly associated with the surface of the sensing electrode and interact directly with the surface of the sensing electrode. In some embodiments, the oxidized sample analytes can be indirectly associated with the surface of the sensing electrode. In such embodiments, the oxidized sample analyte can be associated with one or more sensor analyte which is directly associated with the surface of the sensing electrode, or with one or more oxidized sample analyte which is directly associated with the surface of the sensing electrode. In some specific embodiments, the oxidized sample analyte can be integrated into a dimer, an oligomer or a polymer of one or more species of the sensor/oxidized sample analytes in which at least one monomeric unit is directly associated with the surface of the sensing electrode. In an embodiment, the sensor causes the oxidation of less than about 10%, between about 10 to 50%, 50 to 90% or 90 to 100% of the sample analyte present in the sample.

The oxidation reaction of the sample/sensor analyte is shown in reaction scheme 1 below. Groups R₁, R₂, R₃, R₄, and R₅ can each independently be (i) H, oxide, hydroxide, carboxyl, carbonyl, thiol, nitrogen (secondary or tertiary), (ii) alkyl (including branched and cyclic, optionally combining R groups into the same cyclic group) that is substituted or unsubstituted and may be interrupted by one or more of O, N, and S, or (iii) any other biologically compatible functional group. The oxidation occurs under an applied potential but may occur at low rates if the sample/sensor analyte is exposed to oxidizing species (such as ambient air containing oxygen).

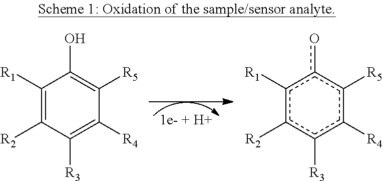

The plurality of sensor analytes covers at least in part the surface of the sensing electrode. In an embodiment, the plurality of sensor analytes covers at least 10, 20, 30, 40, 50, 60, 70, 80, 90% or more of the surface of the sensing electrode. In an embodiment, the plurality of sensor analytes covers a majority or a totality of the sensing electrode's surface. In one embodiment, the entire surface of the sensing electrode is covered by the plurality of sensor analytes. In a specific embodiment, at least about 90, 95, 96, 97, 98, 99% or more of the surface of the sensing electrode is covered by the plurality of sensor analytes.

The sensor analytes can be associated with the sensing electrode with any appropriate technique. The technique should limit or avoid altering the ability of the sensor analytes, once associated with the surface of the sensing electrode, to facilitate the oxidation of the sample analyte. The technique should limit or avoid altering the electrochemical features (such as the conductivity) of the sensing electrode. In one embodiment, the technique is electrodeposition to obtain a plurality of electrodeposited sensor analytes.

The sensing electrode can be made from any suitable conductive material. In one embodiment, the sensing electrode comprises a carbon-based material, a nanomaterial, a metal-based material, or a combination thereof. In one embodiment, the sensing electrode comprises carbon, gold, platinum, palladium, ruthenium, rhodium, or a combination thereof. In a further embodiment, the sensing electrode may be a screen-printed electrode (SPE). The sensing electrode may be of any shape or size. Known SPE includes, but is not limited to, a Zensor electrode, a Dropsens electrode, a Zimmer Peacock electrode, Flex Medical Electrode or a Kanichi electrode. In one embodiment, the sensing electrode is a Zensor carbon-based electrode.

The sensor analyte, prior to its association with the sensing electrode, can correspond to the same chemical species as the sample analyte (prior to detection). The sensor analyte, prior to its association with the sensing electrode, and the sample analyte, prior to detection, can have an oxidizable phenolic group. Furthermore, the sensor analyte, prior to its association with the sensing electrode, and the sample analyte, prior to detection, may be a small molecule having a molecular weight of less than about 5,000 g/mol, less than about 4,000 g/mol, less than about 3,000 g/mol, less than about 2,000 g/mol, or less than about 1,000 g/mol. In one embodiment, the sensor/sample analyte contains at least two fused cyclic structures. In one embodiment, the sensor/sample analyte contains at least three fused cyclic structures. In one embodiment, the sensor/sample analyte contains exactly three fused cyclic structures. In a further embodiment, the three cyclic structures are 6 atom (hexa) rings. The sample/sensor analyte can be, without limitation, a cannabinoid, an opiate, a neurotransmitter, a hormone, or a derivative thereof.

The cannabinoid can be, for example, Δ⁹-tetrahydrocannabinol (THC), 11-hydroxy-Δ⁹-tetrahydrocannabinol (11-hydroxy-THC), delta-8-tetrahydrocannabinol (Δ8-THC), 11-nor-9-carboxy-tetrahydrocannabinol (11-nor-9-carboxy-THC), cannabidiol (CBD), cannabinol (CBN), and glucuronic acid conjugated COOH-THC (gluc-COOH-THC), tetrahydrocannabinolic acid (THCA) or metabolites thereof. The opiate can be, for example, morphine, hydromorphone and buprenorphine as well as metabolites thereof. The neurotransmitter can be, for example, dopamine, serotonin, or metabolites thereof. The hormone can be, without limitation, a steroid hormone such as, for example, estradiol, 7α-methylestradiol, or metabolites thereof.

In some embodiments, the sensor analytes, prior to their association with the sensing electrode, can be THC. In further embodiments, the plurality of sensor analytes associated with the sensing electrode can comprise one or more (in any proportion) different chemical species comprising THC or derived from the oxidation of THC. Chemical species derived from the oxidation of THC include, without limitation, an oxidized form of THC, a dimer of oxidized THC, an oligomer of oxidized THC and/or a polymer of oxidized THC. The plurality of sensor analytes can include a first chemical species corresponding to the THC having an oxidizable phenolic group. In such embodiments, the sensor analyte and the sample analyte (prior to detection) correspond to the same chemical species. The plurality of sensor analytes can include a second chemical species corresponding to the THC oxidized at the oxidizable phenolic group. The plurality of sensor analytes can include a third chemical species corresponding to a dimer, an oligomer or a polymer of the THC oxidized at the oxidizable phenolic group. The plurality of sensor analytes on the sensing electrode can comprise, in any proportion, the first, second, and third chemical species including THC or derived from THC described herein. In a specific embodiment, the plurality of sensor analytes comprises the first and the second chemical species described herein. In a specific embodiment, the plurality of sensor analytes comprises the second and third chemical species described herein. In a specific embodiment, the plurality of sensor analytes comprises the first and the third chemical species described herein. In a specific embodiment, the plurality of sensor analytes comprises the first, the second, and the third chemical species described herein.

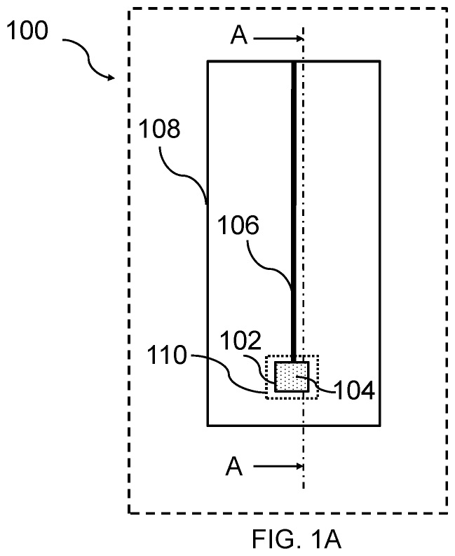

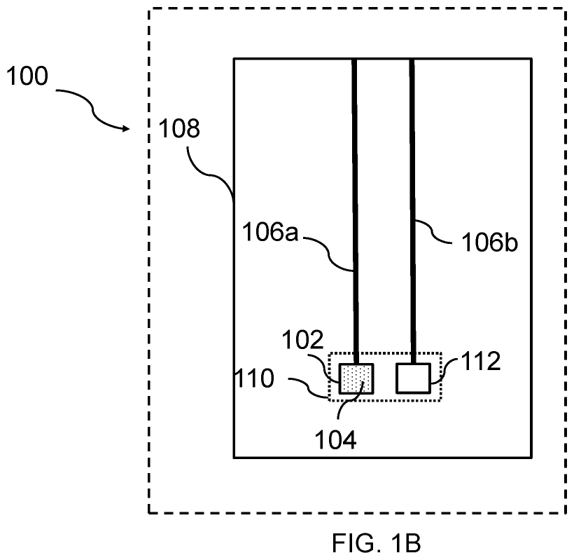

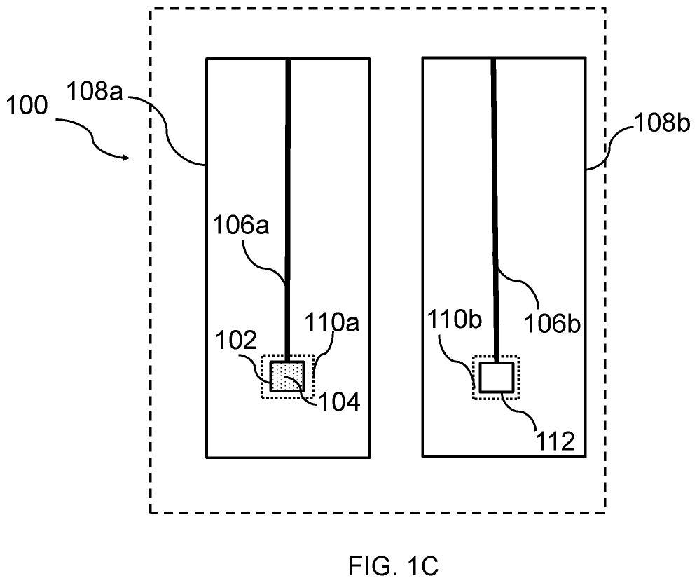

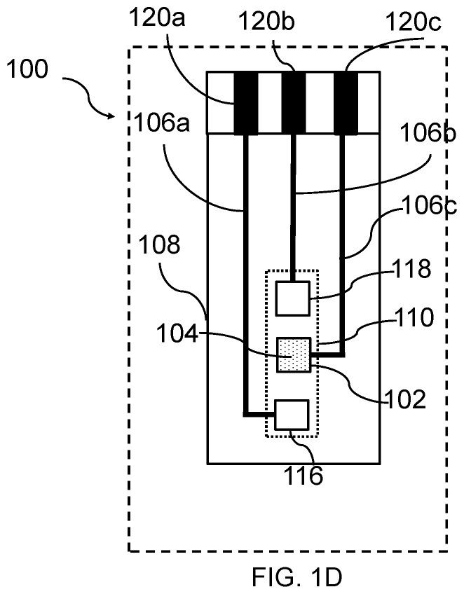

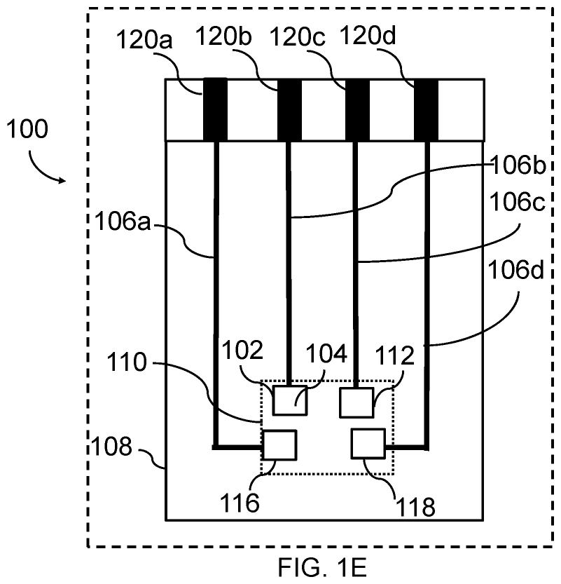

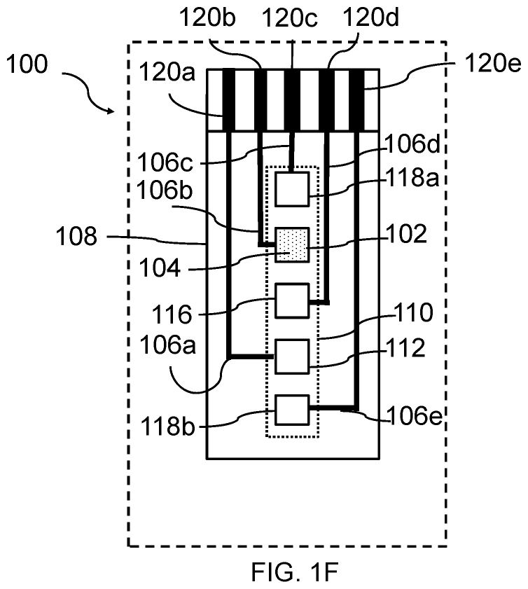

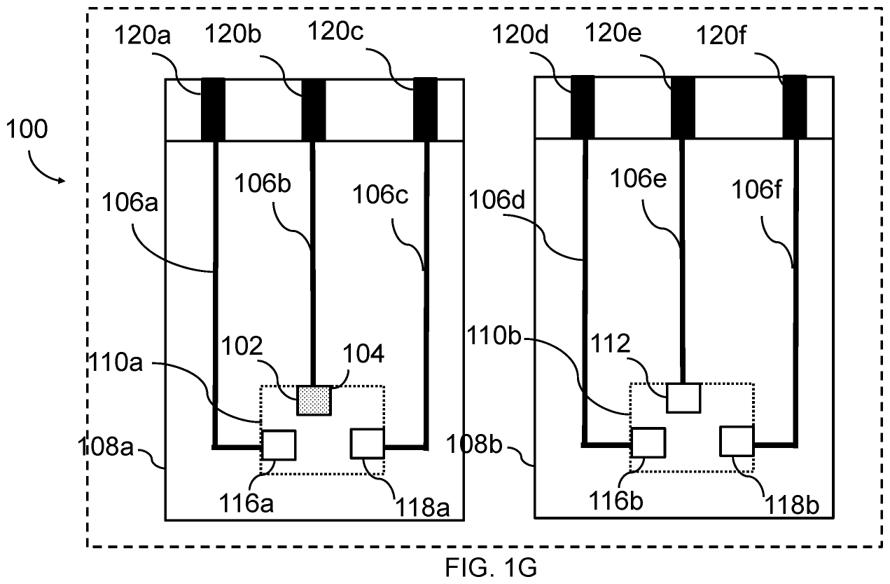

An embodiment of a sensor 100 comprising a sensing electrode 102 is shown in FIG. 1A. In the embodiment presented in this figure, the sensing electrode 102 is associated with a plurality of sensor analytes 104. The sensing electrode 102 can be provided on a substrate 108. The substrate 108 can be an insulated substrate. It is possible that the sensing electrode 102 be self-supporting and as such the substrate 108 may be omitted. A connection 106 connects the sensing electrode 102 to the edge of the substrate 108 or to a contact surface or connecting pad (not shown in FIG. 1A). In the sensor 100 shown in FIG. 1A, a sample receiving region 110 is in fluid communication with the sensing electrode 102. For example, the sample region 110 may be defined to allow contact between the sample and the sensing electrode 102. It will be appreciated that the sample receiving region 110 does not need to cover the sensing electrode 102, in part or in whole (as shown in FIG. 1A), as other configurations for providing fluid communication between the sample receiving region 110 and the sensing electrode 102 can be used (a microfluidic channel for example).

In some embodiments, the sensor comprises a baseline electrode. As used in the context of the present disclosure, the baseline electrode is a working electrode designed to detect and optionally quantify the contribution of electroactive agents present in the sample which can interfere with the detection of the sample analyte. The baseline electrode corresponds to the sensing electrode prior to its association with the plurality of sensor analytes. In some embodiments, the baseline electrode is a bare working electrode.

The baseline electrode can include any suitable conductive material and can be made of the same material as the sensing electrode (without the sensor analytes). In one embodiment, the baseline electrode comprises a carbon-based material, a nanomaterial, a metal-based material, or a combination thereof. In one embodiment, the baseline electrode comprises carbon, gold, platinum, palladium, ruthenium, rhodium, or a combination thereof. In a further embodiment, the baseline electrode may be a screen-printed electrode (SPE). The baseline electrode may be of any shape or size. Known SPE include, but are not limited to, a Zensor electrode, a Dropsens electrode, a Zimmer Peacock electrode, Flex Medical Electrode or a Kanichi electrode. In one embodiment, the basaline electrode is a Zensor carbon-based electrode.

Another embodiment of the sensor 100 is shown in FIG. 1B. In the embodiment provided in FIG. 1B, the sensor 100 comprises the sensing electrode 102 and a baseline electrode 112. The sensing electrode 102 is associated with the plurality of sensor analytes 104. The sensing electrode 102 and the baseline electrode 112 can be provided on the substrate 108. The substrate 108 can be an insulated substrate. It is possible that the sensing electrode 102 and/or the baseline electrode 112 be self-supporting and as such the substrate 108 may be omitted. A connection 106a connects the sensing electrode 102 to the edge of the substrate 108 or to a contact surface or connecting pad (not shown in FIG. 1B). A connection 106b connects the baseline electrode 112 with the edge of the substrate 108 or to a contact surface or connecting pad (not shown in FIG. 1B). In the sensor 100 shown on FIG. 1B, a sample receiving region 110 is in fluid communication with the sensing electrode 102 and the baseline electrode 112. For example, the sample receiving region 110 may be defined to allow contact between the sample and both the sensing electrode 102 and the baseline electrode 112. In some embodiments, distinct sample receiving regions may be provided for each sensing electrode 102 and the baseline electrode 112 (not shown in FIG. 1B). It will be appreciated that the sample receiving region 110 does not need to cover the sensing electrode 102 and the baseline electrode 112 in part or in whole (as shown in FIG. 1B) as other configurations for providing fluid communication between the sample receiving region 110, the sensing electrode 102, and the baseline electrode 112 can be used (a microfluidic channel for example).

Another embodiment of the sensor 100 is shown in FIG. 1C. In the embodiment provided in FIG. 1C, the sensing electrode 102 and baseline electrode 112 are provided on separate substrates 108a, 108b, respectively. The distinct sample receiving regions 110a and 110b are provided for each of the sensing electrode 102 and the baseline electrode 112, respectively. It will be appreciated that the sample receiving regions 110a, 110b do not need to cover the sensing electrode 102 and the baseline electrode 112, in whole or in part.

In some embodiments, the sensor includes one or more reference electrodes. In an embodiment, each working electrode (i.e. the sensing electrode 102 and the baseline electrode 112) can be associated with one or more reference electrodes. In another embodiment, two or more working electrodes can be associated with the same reference electrode. The reference electrode is an electrode with a stable and well-defined electrochemical potential against which the potential of other electrodes like the sensing electrode or baseline electrode can be controlled and measured. When the reference electrode is in use, it is intended to be covered by the sample. In one embodiment, the reference electrode comprises or consists of silver. When the reference electrode is screen printed, it can be prepared with Ag/AgCl ink or Ag ink.

In some embodiments, the sensor includes one or more counter electrode. In an embodiment, each working electrode can be associated with one counter electrode. In another embodiment, two or more working electrodes can be associated with the same counter electrode. The counter electrode completes the circuit of a three-electrode cell, as it allows the passage of current. After the sample is placed on a sample receiving region, a potential is applied between the sensing electrode and the reference electrode, and the current induced is measured. At the same time, a potential between the counter electrode and the reference electrode is induced which will generate the same amount of current (reverse current). Therefore the sensing electrode, baseline electrode, reference electrode, and counter electrode are all intended to be in fluid communication with the sample. The counter electrode can be made of the same materials as the sensing electrode and/or the baseline electrode and/or the reference electrode. In one example, the counter electrode comprises or consists of carbon ink or platinum.

Another embodiment of the sensor 100 is shown in FIG. 1D. In the embodiment provided in FIG. 1D, the sensor 100 comprises a sensing electrode 102, a reference electrode 116 and a counter electrode 118. The sensing electrode 102 is associated with a plurality of sensor analytes 104. The sensing electrode 102, the reference electrode 116 and the counter electrode 118 are provided on the same substrate 108. The substrate 108 can be insulated. It is understood that any of the electrodes of the sensor 100 can be self-supporting and do not need to be provided on the substrate 108. A connection 106c connects the sensing electrode 102 to a contact surface 120c. A connection 106a connects the reference electrode 116 to a contact surface 120a. A connection 106b connects the counter electrode 118 to a contact surface 120b. A common sample receiving region 110 is provided for all of the electrodes 102, 116, 118. It will be appreciated that the sample receiving region does not need to cover the regions defined by the electrodes, in part or in whole, as other configurations for providing the sample to the electrodes 102, 116, 118 can be designed (a microfluidic channel for example). Distinct sample receiving regions can also be provided for each electrode 102, 116, 118.

Another embodiment of the sensor 100 is shown in FIG. 1E. In the embodiment provided in FIG. 1E, the sensor 100 comprises a sensing electrode 102, a baseline electrode 112, a reference electrode 116 and a counter electrode 118. The sensing electrode 102 is associated with a plurality of sensor analytes 104. The sensing electrode 102, the baseline electrode 112, the reference electrode 116 and the counter electrode 118 are provided on a same substrate 108. The substrate 108 can be insulated. It is understood that any of the electrodes of the sensor 100 can be self-supporting and do not need to be provided on the substrate 108. A connection 106b connects the sensing electrode 102 to a contact surface 120b. A connection 106c connects the baseline electrode 112 to a contact surface 120c. A connection 106a connects the reference electrode 116 to a contact surface 120a. A connection 106d connects the counter electrode 118 to a contact surface 120d. A common sample receiving region 110 is provided for all of the electrodes 102, 112, 116, 118. It will be appreciated that the sample receiving region does not need to cover the regions defined by the electrodes, in part or in whole, as other configurations for providing the sample to the electrodes 102, 112, 116, 118 can be designed (a microfluidic channel for example). Distinct sample receiving regions can also be provided for each electrode 102, 112, 116, 118.

Another embodiment of the sensor 100 is shown in FIG. 1F. In the embodiment provided in FIG. 1F, the sensor 100 comprises a sensing electrode 102, a baseline electrode 112, a reference electrode 116 and counter electrodes 118a and 188b. The sensing electrode 102 is associated with a plurality of sensor analytes 104. The sensing electrode 102, the baseline electrode 112, the reference electrode 116 and the counter electrodes 118a and 188b are provided on a same substrate 108. The substrate 108 can be insulated. It is understood that any of the electrodes of the sensor 100 can be self-supporting and do not need to be provided on the substrate 108. A connection 106b connects the sensing electrode 102 to a contact surface 120b. A connection 106a connects the baseline electrode 112 to a contact surface 120c. a A connection 106d connects the reference electrode 116 to a contact surface 120d. A connection 106a connects the counter electrode 118a to a contact surface 120c. A connection 106e connects the counter electrode 118b to a contact surface 120e. A common sample receiving region 110 is provided for all of the electrodes 102, 112, 116, 118a, 188b. It will be appreciated that the sample receiving region does not need to cover the regions defined by the electrodes, in part or in whole, as other configurations for providing the sample to the electrodes 102, 112, 116, 118a, 188b can be designed (a microfluidic channel for example). Distinct sample receiving regions can also be provided for each electrode 102, 112, 116, 118a, 188b.

Another embodiment of the sensor 100 is shown in FIG. 1G. In this example, the sensing electrode 102 and baseline electrode 112 are provided on separate substrates 108a, 108b, respectively. Each of the susbtrates 108a and 108b comprise a reference electrode 116a, 116b and a counter electrode 118a, 118b. The electrodes 102, 116a, 118a are connected to contact surfaces 120a, 120b, 120c by connections 106a, 106b, 106c. The electrodes 112, 116b, 118b are connected to contact surfaces 120d, 120e, 120f by connections 106d, 106e, 106f. Each substrate 108a, 108b, has a sample receiving region 110a, 110b in fluid communication with corresponding electrodes.

In some embodiments, the substrate(s) can support a dielectric layer surrounding, at least in part, one electrode of the sensor. In specific embodiments, the substrate(s) can support a dielectric layer surrounding all of the electrodes of the sensor.

In some embodiments, the sensor as described herein is used for the detection of the sample analyte in a sample using a voltammetry technique. Voltammetry techniques are electroanalytical techniques based on the detection and quantification of an analyte, by measuring a current as an applied potential is varied. In one embodiment, the voltammetry techniques are, but not limited to, cyclic voltammetry (CV), linear sweep voltammetry (LSV), differential pulse voltammetry (DPV), or square wave voltammetry (SWV). CV is performed by cycling the potential of a working electrode (e.g., the sensing and the baseline electrodes) ramped linearly versus time, and measuring the resulting current. LSV measures the current at the working electrodes (e.g., the sensing and the baseline electrodes) while the potential between the working electrode and a reference electrode is swept linearly in time. In the DPV technique a potential scan is recovered by imposing potential pulses with a constant amplitude. The differences between the currents registered just before and at the end of the pulse are plotted versus the potential. SWV is a large-amplitude differential technique in which a waveform composed of a symmetrical square wave, superimposed on a base staircase potential, is applied to the working electrodes (e.g., the sensing and the baseline electrodes).

The sensor can be used for the detection of a sample analyte suspected to be present in a sample comprising electroactive agents that can interfere with the detection of the sample analyte. The electroactive agents can be, without limitations, an organic component (such as for example uric acid, glutamate, ascorbic acid or a combination thereof), an inorganic component (such as, for example a salt which can be, without limitation, NaCl, NH₄Cl, NaH₂PO₄, KCl, Na₃Cit, MgCl₂, Na₂CO₃, CaCl₂ or a combination thereof) and/or a biological component (such as, for example, a protein which can be, without limitation, albumin, lysozyme, mucin or a combination thereof). The sample can be a biological sample which can be, without limitation, any bodily fluid, such as an oral fluid. The sample can also be obtained from an edible product (such as food or a beverage). The sample can be obtained from a drug (legal or illegal).

The sensor can be packaged during storage or transport. It can be placed in an inert environment or vacuum-sealed. The packaging may be useful to preserve the stability of the electrode and limit or advantageously avoid any species that would produce an oxidation reaction. For example, the inert environment can comprise or consist of nitrogen, helium, argon, or combinations thereof. In one embodiment, the packaging is free of any containing gases such as oxygen and carbon dioxide. In an embodiment, the package comprising the sensor includes a temperature, an oxygen and/or a moisture sensor to determine the storage conditions and provide information on the potential oxidation of the sensor analytes.

The sensor can include additional working electrodes, for example for the detection of other target analytes which are suspected to be present in the sample or in another sample. The sensor can be used in combination with other detection techniques to confirm and/or quantify the presence of the sample analyte in the sample.

The sensor can be adapted to receive a sample collection means to provide the sample to the electrodes.

### Processes for Making the Sensor

The sensor can be fabricated using any process capable of associating the plurality of sensor analytes with a sensing electrode. The sensing electrode may be polished and/or washed before the association with the plurality of sensor analytes. The sensing electrode may be washed after the association with the plurality of sensor analytes. In an embodiment, the process also causes, at least in part, the oxidation of the oxidizable group of a portion of the sensor analytes.

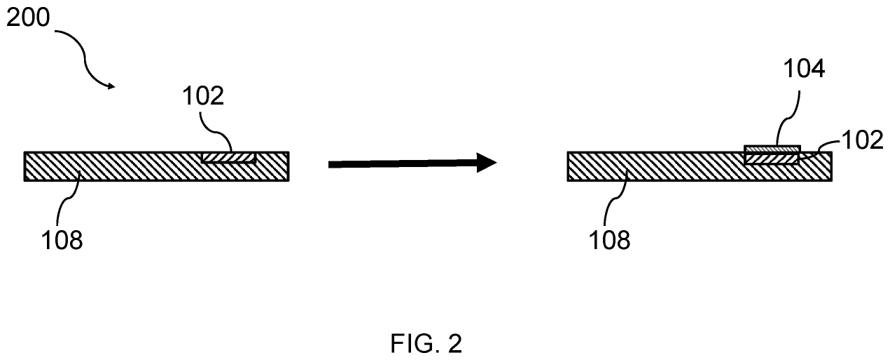

FIG. 2 illustrates an example of a process 200 for making the sensor. The sensing electrode 102 is provided on a substrate 108. However, it is understood that the sensing electrode 102 can be self-supporting and therefore the substrate 108 may be omitted. The sensing electrode 102 is contacted with a plurality of sensor analytes 104 (which can, in some embodiments, be provided in a solution). The plurality of sensor analytes 104 is then associated with the sensing electrode 102 by any process known in the art for associating an analyte with a sensor.

The solution comprising the plurality of sensor analytes can include an aqueous solvent, an organic solvent, or a mixture of both. In some embodiments, the solution comprising the plurality of sensor analytes comprises an organic solvent, such as, for example, methanol, ethanol, or a mixture thereof. In yet a further embodiment, the solution comprising the plurality of sensor analytes comprises a mixture of water and methanol in any ratio, for example, between 1:10 and 10:1 (v/v). In some embodiments, process 200 includes providing the solvent required for making the solution and/or dissolving the plurality of sensor analytes 104 in the solvent to make the solution. In some embodiments, the process 200 can include providing the solution.

The solution that may be provided on the surface of the sensing electrode can be dried before the sensor analytes 104 are associated with the sensing electrode 102. When the solvent is volatile, the solution can be dried at room temperature, optionally in the presence of an airflow. It is possible to use elevated temperatures (e.g., higher than room temperature) as long as the temperatures do not cause substantial oxidation of the sensor analytes and/or do not negatively affect the functioning of the sensing electrode 102. In some embodiments, the process 200 can include drying the solution that has been provided on the surface of the sensing electrode before associating the sensor analytes 104 with the sensing electrode 102.

In an embodiment, the sensor analytes 104 can be associated with the sensing electrode 102 by electrodeposition. In embodiments in which electrodeposition is used to associate the sensor analytes 104, the sensing electrode 102 can receive an electrolytic solution (which can be, without limitation, a buffer, such as, for example a phosphate buffered saline). In some embodiments, the process 200 can include contacting the sensing electrode 102 with an electrolytic solution.

In some embodiments, the sensor analytes 104 can be associated with the sensing electrode 102 by applying at least one potential to the electrode and as such, the process 200 can include applying at least one potential to the sensing electrode 102 in contact with the solution. Application of solution onto the sensing electrode can be performed by manual pipetting, robotic pipetting, inkjet dispensing or using any other appropirate type of liquid dispensing technology. In a specific embodiment, the process 200 can include applying a plurality of potentials (e.g., a potential scan) to the sensing electrode 102 in contact with the solution. In still another embodiment, the process 200 can include applying a voltammetry technique to the sensing electrode 102 in contact with the solution. In one embodiment, the voltammetry technique includes, but is not limited to, cyclic voltammetry (CV), linear sweep voltammetry (LSV), differential pulse voltammetry (DPV), or square wave voltammetry (SWV). In one embodiment, the voltammetry technique is square wave voltammetry (SWV).

In some embodiments, the process 200 can include determining a contribution to the detection process from the plurality of sensor analytes associated with the sensing electrode which still has an oxidizable phenolic group. This contribution can be measured as an electric current (I₀), that occurs when submitting the sensing electrode, in the absence of the sample, to one or more further potentials and measuring the current associated thereto.

The sensing electrode intended to be modified to obtain the sensor can be used without pre-treatment. However, in some embodiments, the sensing electrode can be polished and/or washed prior to the association of the sensor analytes. As such, the process can include polishing and/or washing the surface of the sensing electrode before it is associated with the sensor analytes to obtain the sensor. The polishing step can include mechanical polishing, chemical-mechanical polishing or a combination of both. Polishing can be useful, in some embodiments, to reduce or eliminate any polymeric residues on the electrode surface, which can further improve the electrochemical performance of the electrode. The washing step can include using water (which may be distilled or Milli-Q water).

Once the sensor analytes have been associated with the sensing electrode, the sensor can optionally be treated (and in some embodiments washed) to remove sensor analytes which have not or have loosely associated with the sensing electrode. As such, the process can include washing the sensing electrode after a portion of the sensor analytes have been associated thereto. The washing can include using water (distilled or Milli-Q) or an aqueous solution.

Alternatively or in combination, the sensing electrode, after its association with the plurality of sensor analytes, be further modified to include a coating that is capable of preventing or limiting the oxidation of the plurality of the sensor analyte. The coat can be, for example, a solution. In another embodiment, the coat can be dissolved in the presence of an aqueous solution, such as, for example, saliva.

As indicated above, the baseline electrode does not include the sensor analytes and as such does not need to be treated before being used in the sensor. However, the baseline electrode can be treated before being used in the sensor. For example, in some embodiments, the baseline electrode is a bare working electrode that can be polished and/or washed. As such, the process can include polishing and/or washing the surface of the baseline electrode. The polishing step can include mechanical polishing, chemical-mechanical polishing or a combination of both. Polishing can be useful, in some embodiments, to reduce or eliminate any polymeric residues on the electrode surface, which can further improve the electrochemical performance of the electrode.

The washing step can include using water (which may be distilled or Milli-Q water). In some embodiments, the baseline electrode is polished/washed under the same conditions as the sensing electrode (prior and/or after the association with the sensor analytes).

The sensor can be stored at cold temperature (not freezing, for example 4° C.) before use. As such, the process can include storing the sensor prior to use.

The sensor can be packaged to limit or prevent oxidation of the sensor analytes. The package comprising the sensing electrode can also include the baseline electrode. Alternatively, the sensing electrode and the baseline electrode can be packaged separately. As such, in some embodiments, the process can include packaging the sensing electrode and/or the baseline electrode. The package can create an inert environment for the electrodes. For example, the inert environment can comprise or consist of nitrogen, helium, argon, or combinations thereof. In one embodiment, the packaging is free of any containing gases such as oxygen and carbon dioxide. The package can be vacuum-sealed.

### Detection System and Computing Devices

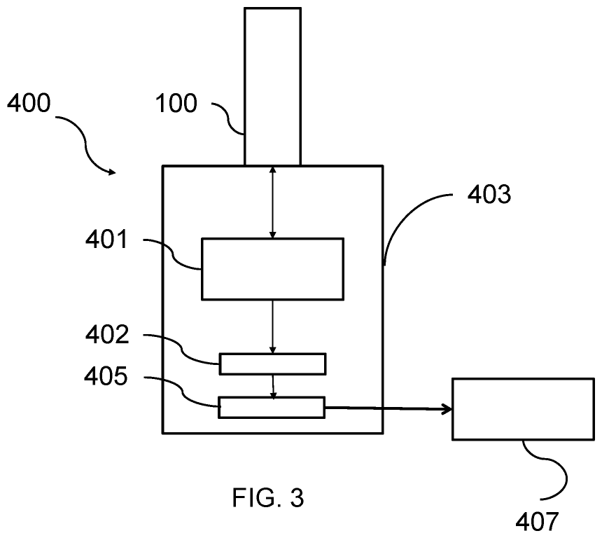

With reference to FIG. 3, the sensor 100 may be used with a computing device 403 to form a detection system 400, which may be handheld, portable, or fixed. For simplicity only one computing device 403 is shown but a detection system 400 may include more computing devices 403 operable by users to access remote network resources and exchange data. The computing devices 403 may be the same or different types of devices. The computing device 403 comprises at least one processor 401, a data storage device 402 (including volatile memory or non-volatile memory or other data storage elements or a combination thereof), and at least one communication interface 405. The computing device 403 components may be connected in various ways including directly coupled, indirectly coupled via a network, and distributed over a wide geographic area and connected via a network (which may be referred to as “cloud computing”). It will be understood that the computing device 403 comprises all analog circuitry necessary to interface with the sensor 100.

For example, and without limitation, the computing device 403 may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein.

Each processor 401 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof. In some embodiments, the processor 401 is embodied as a potentiostat.

Memory 402 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each communication interface 405 enables computing device 403 to interconnect with one or more input/output devices 407, such as a keyboard, mouse, camera, touch screen microphone, display screen and speaker. For example, a display screen may display a symbol or sign that is indicative of the presence or absence of the sample analyte in the sample. In one embodiment, the display screen displays the value of the concentration of the sample analyte in the sample. In a further embodiment, the display screen may display the estimated value of the concentration of the sample analyte in the sample of the subject who provided the sample. The display can be a simple display in black and white or a more modern touch screen able to receive commands. A user interface may contain a button or other physical means for the user to signal to the device to begin the analysis of a sample.

In some embodiments, a network interface enables computing device 403 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 403 can be operable to register and authenticate users (using a login, unique identifier, and password for example) prior to providing access to applications, a local network, network resources, other networks and network security devices. Computing device 403 may serve one user or multiple users.

The sensor can be provided as part of a kit with or without the computing device 403. The kit can include one or more of a sensing electrode, instructions to perform the methods described herein, optionally a calibration sample, and optionally a blank sample.

### Methods of Using the Sensor for Detecting a Sample Analyte

The sensor of the present disclosure is intended to be used to detect a sample analyte suspected to be present in a sample. The sample can be a complex one as it can include one or more electroactive agents which can limit the detection of the sample analyte. The sample can be a biological sample. The sample can be an edible product such as a food sample, and/or a beverage sample. The sample can be a drug sample. The electroactive agents that can be present in the biological sample can include, without limitations, an organic component (such as for example uric acid, glutamate, ascorbic acid or a combination thereof), an inorganic component (such as, for example a salt which can be, without limitation, NaCl, NH₄Cl, NaH₂PO₄, KCl, Na₃Cit, MgCl₂, Na₂CO₃, CaCl₂ or a combination thereof) and/or a biological component (such as, for example, a polynucleotide or a protein which can be, without limitation, albumin, lysozyme, mucin or a combination thereof). The sample can be a biological sample which can be, without limitation, an ex vivo bodily fluid that can be a non-invasively obtained fluid (saliva, sputum, urine, tears, etc.) or invasively obtained (blood, plasma, cerebral spinal fluid, etc.). In an embodiment, the bodily fluid is an oral fluid. The oral fluid can include saliva, sputum, or a combination thereof. The sample can be used with the sensor described herein without being processed (e.g., an unprocessed sample). In some embodiments, the bodily fluid sample can first be processed before being used with the sensor described herein. As such, only some components of the sample can be placed into contact with the electrodes of the sensor described herein. The sample can be obtained from an animal (e.g., a mammal, for example, a human), a plant or a microorganism.

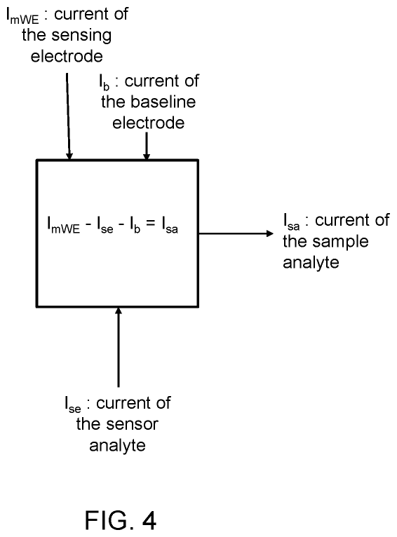

The method described herein relies on the notion that when an electric potential is applied to the sensing electrode having the plurality of sensing analytes associated thereto while in fluid communication with the sample, the resulting measurement contains a contribution from the sample analyte (if present) and a contribution from interfering electroactive agents in the sample. In order to isolate the contribution from the sample analyte, the contribution from the interfering electroactive agents is determined using the baseline electrode and subtracted from the measurement obtained from the sensing electrode. This is illustrated schematically in FIG. 4. In some embodiments, the contribution of the sample analyte in the sample (Iₛₐ) can be represented with equation (1) as follows:

Iₛₐ=I_{mWE}−I_{b} (1).

In equation (1), Iₛₐ is the current contributed from the sample analyte in the sample, I_{mWE} is the current measured at the sensing electrode, and I_{b} is a pre-determined value associated with the current measured at the baseline electrode or the current measured at the baseline electrode.

In some embodiments, the baseline current (I_{b}) is pre-determined. For example, the baseline current (I_{b}) may be stored in the memory 402 of the computing device 403 and retrieved by the processor 401 when performing the detection. In some embodiments, the baseline current (I_{b}) is determined by a separate computing device of the detection system 400 and provided to the computing device 403, either prior to the detection or concurrently therewith. In some embodiments, the baseline current (I_{b}) is measured concurrently with the sensing electrode current (I_{mWE}) by the computing device 403.

In some embodiments, a contribution during the detection process from the oxidation of the sensor analytes associated with the sensing electrode is negligible and thus not considered in determining Iₛₐ. Alternatively, a non-negligible contribution occurs from the oxidation of the sensor analytes associated with the sensing electrode. In this case, the contribution of the sample analyte in the sample (Iₛₐ) can be represented by equation (2) as follows:

Iₛₐ=I_{mWE}−Iₛₑ−I_{b} (2).

In equation (2), Iₛₑ is the contribution from the sensor analytes associated with the sensing electrode and may be considered as a calibration current. The contribution from the sensor analytes (Iₛₑ) may be pre-determined and stored in the memory 402 of the computing device 403 and retrieved by the processor 401 when performing the detection. In some embodiments, the contribution from the sensor analytes (Iₛₑ) is stored remotely and provided to the computing device 403, either prior to the detection or concurrently therewith. Alternatively, the contribution from the sensor analytes may be determined by applying at least one potential to the sensing electrode in fluid contact with the sensor analyte in the absence of the sample, prior to receiving the sample on the sensing electrode.

The sample according to the present disclosure may comprise a bodily (biological) fluid (from a mammal such as a human or another non-mammalian animal). In one embodiment, the sample has a volume that is sufficient to cover the surface of all of the electrodes of a sensor according to the present disclosure. In another embodiment, the sample is diluted with a known amount of a dilutent to cover the surface of all of the electrodes of the sensor. The sample can be a positive sample (comprising a significant amount of sample analyte) or a negative sample (comprising a negligible or trace amount of sample analyte).

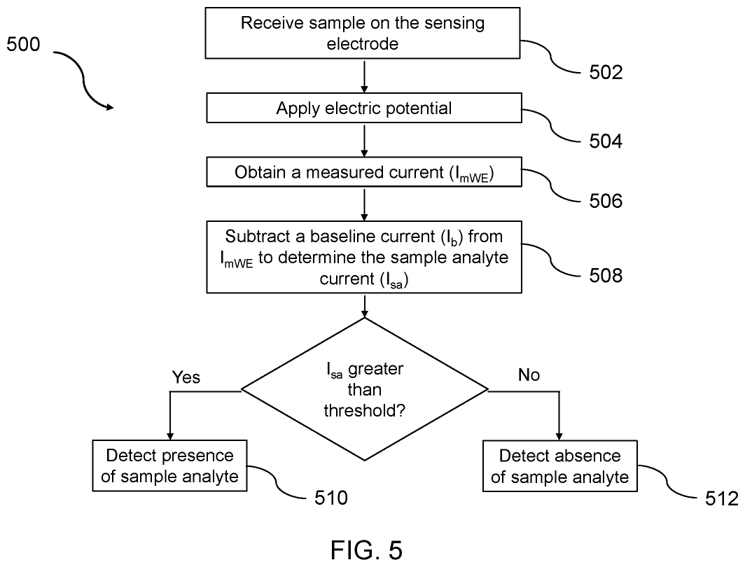

An embodiment of a method 500 of detecting the sample analyte is provided in FIG. 5. In this figure, at step 502, the method comprises receiving a sample on the sensing electrode (by providing, for example, the sample to the sample receiving region of the sensor). When the sensing electrode is in fluid contact with the sample, the method provides, at step 504, applying at least one electric potential to the sensing electrode to oxidize the sample analyte and obtain an oxidized sample analyte. Applying at least one electric potential also causes a portion of the oxidized sample analyte to associate with the surface of the sensing electrode. At step 506, a measured current (I_{b}) is obtained from the baseline electrode concurrently with the application of the at least one electric potential. Steps 504 and 506 are not limited to a single technique and can, for example, be performed using a voltammetric technique (including, but not limited to square wave voltammetry, cyclic voltammetry, linear sweep voltammetry, or differential pulse voltammetry). The voltammetry applied can be programmed to run autonomously and automatically using the processor 401 of the computing device 403. In one embodiment, the voltage applied is a discrete value corresponding to the oxidation peak ±about 10%, ±about 5%, ±about 3%, ±about 2%, ±about 1%. In one embodiment, the voltage applied is one or more potentials in the range of ±0.5 V, ±0.4 V, ±0.3 V, ±0.2 V, ±0.1 V around the peak of oxidation. When a voltammetric technique is used to detect THC as a sample analyte, step 504 of the method 500 can comprise applying a plurality of electric potentials in the range of between 0 to 1 V to the sensing electrode. Once the measured current (I_{mWE}) from the sensing electrode has been obtained at step 506 it is compared to a baseline current to determine a difference at step 508. In the embodiment shown in FIG. 5, the current from the baseline electrode I_{b} is subtracted from the current measured from the sensing electrode (I_{mWE}) at step 508 to determine the sample analyte current (Iₛₐ) (as per equation (1) above).

In some embodiments, the method 500 further comprises obtaining the current from the baseline electrode (I_{b}). The current from the baseline electrode (I_{b}) can be obtained by receiving the sample on a sample receiving region of the baseline electrode, applying at least one electric potential to the baseline electrode and obtaining a measured current from the baseline electrode as the at least one electric potential is applied.

In some embodiments, step 508 comprises taking into account a contribution from the sensor analytes associated with the sensing electrode when determining the sample analyte current (Iₛₐ). In this case, a current corresponding to the sensor analytes (Iₛₑ) is subtracted from the difference between I_{mWE} and I_{b} to obtain Iₛₐ (as per equation (2) above). Iₛₑ can be obtained by a measurement at the sensing electrode in the absence of the sample or can be a pre-determined value retrieved during the detection process. The current obtained from the baseline electrode (I_{b}) can be a pre-determined value or measured during the detection process. The Iₛₐ value obtained at step 508 is compared to a detection threshold to determine if the sample analyte is present (at step 510) or absent (at step 512) in the sample.

In some embodiments, the current determined to be from the sample analyte (Iₛₐ) is proportional to a concentration of the analyte in the sample and is calculated using a mathematical function obtained from a calibration curve.

In some embodiments, the method 500 comprises quantifying the amount of analyte in the sample. For example, a correspondence table may be used to determine a quantity as a function of the value of Iₛₐ. An example embodiment is shown in Table 1 below.

TABLE 1

| Iₛₐ (mA) | Analyte Quantity (ng/mL) |
|---|---|
| X₁ | Y₁-Y₂ |
| X₂ | Y₃-Y₄ |
| X₃ | Y₅-Y₆ |

In Table 1, values for Iₛₐ current are associated with ranges of analyte quantity. In some embodiments, ranges of Iₛₐ current values are associated with single values for analyte quantity. In some embodiments, ranges of Iₛₐ current values are associated with ranges of analyte quantity. It will be readily understood that various embodiments may be used to quantify the amount of analyte in the sample as a function of the Iₛₐ current as determined by the method 500. Any relationship found between the current determined to be from the sample analyte (Iₛₐ) and the concentration of the analyte in the sample may be used in a look-up table or other format to quantify the amount of analyte in the sample.

The method of detection described herein can achieve, depending on the sample analyte being tested and the embodiments of the sensor, a limit of detection equal to or less than 100, 90, 80, 70, 60, 50, 40, 30, 20, 10, 5, 1 ng/mL of the sample analyte in the sample. In embodiments in which the sample analyte is THC and the sample comprises saliva, the method of detection described herein can achieve a limit of detection equal to or less than 50, 25, 10, 9, 8, 7, 6, 5, 4, 3, or 2 ng/mL of THC in the saliva sample.

When the sample intended to be used is saliva, the sensor can be placed in the oral cavity of the subject to facilitate the contact of the subjects's bodily fluid (saliva in this embodiment) with the sample receiving region of the sensor. In an embodiment, the sensor can be placed in the vicinity of the tongue or mandible of the subject and can even, in some further embodiments, be placed in contact with the subject's tongue or mandible (to gather, for example, submandibular and/or sublingual saliva). Alternatively or in combination, the sample can be obtained from a collecting means and then received on the sample receiving region of the sensor. The collecting means may include collecting saliva by placing a porous filter media into the subject's oral cavity, which absorbs saliva found in the oral cavity, and subsequently expressing the saliva onto the sensor.

Alternately, saliva can be collected by a subject expectorating into a container and subsequently transferring a sufficient volume of saliva onto the sensor. In an embodiment, a sufficient volume of the sample is such that the sample covers the surface of the sensing electrode and the baseline electrode. In an embodiment, the volume of the sample received in the sample-receiving region is between about 50 μL to about 1 mL. These values may vary depending on practical implementations.

When the sample intended to be used is a solid material, it can be first dissolved in a solvent (such as water) prior to place it on the sample receiving region of the sensor.

In the detection methods described herein, the plurality of sensor analytes associated with the sensing electrode facilitates, in the presence of an electric potential, the oxidation of the oxidizable phenolic group of the sample analyte to generate an oxidized sample analyte. A portion of the oxidized sample analyte will be associated with the sensing electrode. The oxidized sample analytes can be, at least in part, directly associated with the sensing electrode by directly interacting with the surface of the sensing electrode. In some embodiments, the oxidized sensor analytes can be indirectly associated with the surface of the sensing electrode. In such embodiments, the oxidized sensor analytes can be associated with one or more sensor analyte which is directly associated with the surface of the sensing electrode or with one or more oxidized sample analyte which is directly associated with the surface of the sensing electrode. In some specific embodiments, the oxidized sample analyte is integrated in a dimer, an oligomer or a polymer of one or more species of the sensor/oxidized sample analytes in which at least one monomeric unit is directly associated with the surface of the sensing electrode. In some embodiments, the majority or the totality of the sample analyte is oxidized during the detection.

In one embodiment, the sample is a biological sample (such as a bodily fluid) that is obtainable non-invasively (e.g., an ex vivo bodily fluid). For example, the sample can be an oral fluid sample such as saliva or sputum, a lavage, or an epithelial swab of an individual's tissue. Other examples of non-invasive bodily fluids include but are not limited to urine, sweat, stools, and tears. In some embodiments, the sample may be a bodily fluid that is obtainable invasively such as blood, plasma, or cerebral spinal fluid. In one embodiment, the sample has been obtained from an animal, such as a mammalian animal (a human for example), a plant or a microorganism. As such, the method of detection described herein can include a step of obtaining a sample from a subject (which can be an animal, a plant or a microbe).

In some embodiments, the sample can be used without prior treatment and received on the sample receiving region of the sensor. In other embodiments, the sample can be treated before being received on the sample receiving region of the sensor. In such embodiments, a treated sample will be received on the sample receiving region of the sensor. A treated sample comprises a component of a sample and refers to a sample that has been treated before the detection process. The treatment can include, without limitation, the removal of at least one component of the sample (such as solid residues, proteins, polynucleotides, charged entities, and the like), the dilution of the sample, the freezing of the sample or the heat-treatment of the sample. The treatment is to preserve, as much as possible, the integrity (and especially the oxidation state) of the sample analyte.

In a specific embodiment, the sample comprises saliva or a saliva component suspected of comprising the sample analyte. The saliva sample can be provided without any treatment steps and received on the sample receiving region of the sensor. The saliva sample can optionally be treated before being received on the sample receiving region. In one embodiment, the saliva sample can be submitted to a dilution, a filtration, a centrifugation, a precipitation, a pH adjustment or a combination thereof. In a further embodiment, the saliva sample is filtered prior to being received in the sample receiving region of the sensor. In a specific example, a partial filtration can be performed during the collection of saliva with different material, such as, for example swabs made of cotton, cellulose, or synthetic fibers. In another example, the filtration can be performed with a filter having a pore size of between about 0.1 to 0.5 μm or between about 0.1 to 3 μm including filters having diameters between 10 to 30 mm. The filtering membrane includes, but are not limited to, GHP (hydrophilic propylene), hydrophobic PTFE (Polytetrafluoroethylene), PES (Polyethersulfone), hydrophobic PVDF (Polyvinylidene fluoride), nylon, glass wool (trated or untreated), hydrophobic PTFE (Polytetrafluoroethylene), hydrophilic PTFE or any combination thereof. In one embodiment, the pH of the saliva sample can be adjusted by adding a base, an acid or a buffer. In another embodiment, the saliva sample be dissolved in an alcoholic solvent (such as methanol), a buffer (such as phosphate buffer saline), or a combination of both. In one embodiment the ratio of dilution of the saliva sample is between 1:10 to 10:1, between 1:5 to 5:1, between 1.2:1 to 1:1.2, or about 1:1.

### EXAMPLE

Materials and Equipment. The healthy human saliva samples were obtained with consent. TE100 Screen-printed electrodes (SPE) with carbon-based working (3 mm/0.071 cm²), counter and silver reference electrodes were purchased from Zensor R&D. All chemical reagents and proteins (albumin (A7030-10G), lysozyme human (L1667-1G), mucin (M3895-100MG)) were purchased from Sigma-Aldrich. (−)-trans-Δ⁹-tetrahydrocannabinol (THC) standard solution in methanol (1 mg/mL) was purchased from Cerilliant- Sigma-Aldrich with an appropriative certification. ELISA THC Oral Fluid Kit Product No 120519 was purchased from Neogen Corporation. The electrochemical measurements were performed with a PalmSens4 potentiostat driven by the PSTrace 5 software. The bare working (e.g., unmodified, an embodiment of the baseline electrode) and modified working (e.g., modified with THC, an embodiment of the sensing electrode) electrodes were studied using scanning electron microscopy (SEM JSM 7000F) working at 10.0 kV with a LED detector. Raman spectrum was performed with Renishaw InVia Raman Spectrometer with a laser at 635 nm (50% of power) and 20 accumulations scans.

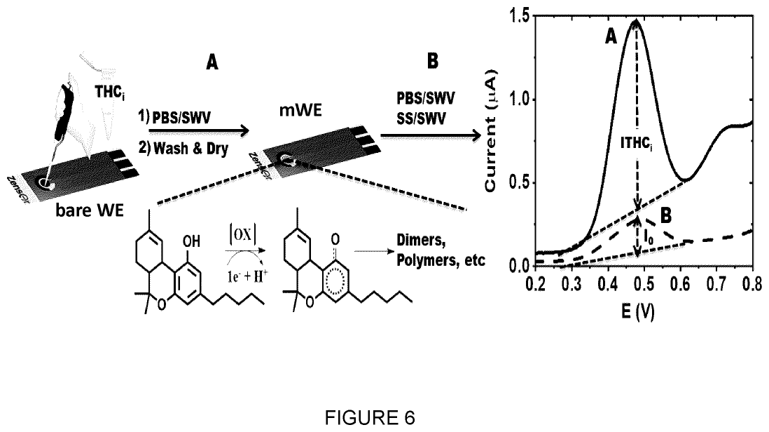

Modification of the electrodes to obtain the modified working electrodes. A schematic representation of how the modified electrodes were obtained is shown in FIG. 6A. First, the working electrodes were thoroughly washed with Milli-Q water and dried with hot airflow. Then, a stock solution of THC (30 μg/mL) was prepared by adding 3 μL of THC (1 mg/mL in methanol) in 97 μL of a mix of solvent methanol/water (3:1 ratio in volume). Immediately after, 1 μL of the previous stock was added to the working electrode. Further, the electrodes were dried at room temperature (RD airflow for 30 seconds and warm airflow for 5 seconds. Afterwards, the dried electrodes received PBS 0.01 M as an electrolyte solution and were submitted to electrochemical treatment by using SWV with the following conditions: precondition potential of 0.05 V for 30 s, equilibration time of 3 s, voltammetric potential scan from 0 to 0.8 V with a frequency of 15 Hz, the amplitude of 25 mV, and step potential of 5 mV. In this case, the intensity of the current is proportional to the amount of THC deposit on the working electrode. This value was registered for each electrode and from now on will be mentioned as ITHCi. After each record, the modified working electrodes were thoroughly washed with Milli-Q water and stored at 4° C. degree in N₂-rich package until used.

In contrast to a common sensor in which a baseline is obtained in the absence of the analyte, in this case, even in the absence of THC in the sample, a peak corresponding to the THC deposited on the modified working electrode is obtained (FIG. 6B). Additionally, because the deposition of THCi resulted in the modified working electrode with a certain degree of variability between electrodes, a number of each batch of modified working electrodes were evaluated during the manufacturing. In this sense, SWV of the sensing in PBS or simulated saliva were registered and a mathematical fit that correlates the current during the deposition (ITHCi) and the current of the new zero (I₀) instead of the baseline was obtained.

Electrochemical characterization. The effects of the modification of the electrodes as well as the media spiked with THC were evaluated by using cyclic voltammetry (CV). In this case, a solution of 100 μL K₄[FeCN₆] (FeCN), 0.1 mM in KCl 0.1M, and FeCN 0.1 mM in simulated saliva (SS) was dropped on the bare and modified working electrodes. Then, CVs were recovered by using different scan rates (0.025 to 0.2 v/s) from −0.3 to 0.6 V. Similar approach was used by adding THC (1 000 ng/mL) in PBS pH 7.4 and SS and then recovered by using different scan rates (0.025 to 0.2 v/s) from 0.2 to 0.8 V.

Sensor operation for THC detection in PBS buffer and simulated saliva. The THC samples were prepared by adding different aliquots of the methanolic stock solutions of THC in the different media of PBS or simulated saliva to obtain final THC concentrations from 1000 to 0 ng/mL. Then, 100 μL of the samples were added on the modified working electrode and, immediately after, SWV was recorded with the following conditions: precondition potential of 0.05 V for 30 s, equilibration time of 3 s, voltammetric potential scan from 0 to 0.8 V with a frequency of 15 Hz, the amplitude of 25 mV, and step potential of 5 mV. Different parameters like electrochemical voltammetric techniques, the influence of the incubation time, and pH were evaluated. The calibration curve plot the difference between the total current intensity regarding the baseline minus the contribution of the THCi as a new zero (I₀) according to the media.

Interferences study. The SWV with the same parameters of the sensor of real saliva and three different solutions containing inorganic, organic, and biological components of saliva were evaluated with the bare and the modified sensing electrode. Table 2 encloses details about the composition of the solution based on the reported levels of such components in saliva. Finally, the simulated saliva (SS) used during the electrochemical studies and in the calibration curve was prepared by mixing inorganic and biological components with the same concentration of table 2.

TABLE 2
Composition of the different solutions evaluated as
possible interferences and the simulated saliva.

| Inorganic | | Organic | | | Biological | | |
|---|---|---|---|---|---|---|---|
| components | Concentration | components | Concentration | | components | Concentration | |
| NaCl | 0.0275M | uric acid | 0.05 | mg/mL | Albumin | 0.2 | mg/mL |
| NH₄Cl | 0.0063M | glutamate | 0.034 | mg/mL | lysozyme | 0.3 | mg/mL |
| NaH₂PO₄ | 0.0049M | ascorbic acid | 1 | ug/mL | mucin | 0.021 | mg/mL |
| KCl | 0.0029M | | | | | | |
| Na₃Cit | 0.0011M | | | | | | |
| MgCl₂ | 0.00002M | | | | | | |
| Na₂CO₃ | 0.0027M | | | | | | |
| CaCl₂ | 0.0002M | | | | | | |

Sensor operation for THC detection in real saliva. After determining the influence of real saliva during the THC detection with the modified working electrode and SWV, slight variations of the protocol were introduced. Real saliva samples were collected by spitting in a container. Then, the saliva was adjusted with different conditions:
the saliva was used without variation and centrifuged (30 seconds, 9000 rpm) to eliminate the solid components. An alternative way to eliminate the solid components was by using a filter with 0.2 μm pore size and GHP membrane (hydrophilic propylene membrane suitable for both aqueous and organic samples).
the saliva was centrifuged (30 seconds, 9000 rpm) to eliminate the solid components and then the pH was adjusted to 8 by adding a small volume of NaOH 0.1M;
the saliva was centrifuged (30 seconds, 9000 rpm) to eliminate the solid components and then diluted (1:1 ration in volume) with PBS pH=7.4.
the saliva was filtered and then diluted with methanol (which also contained the THC to spike the samples) to obtain final saliva: methanol samples with a ratio (9:1).
the saliva was filtered and then diluted with methanol (which also contained the THC to spike the samples) and then with PBS to obtain final saliva:methanol: PBS samples with different ratios.

Known concentrations of THC in methanol were then spiked to the different saliva samples previously obtained ((i), (ii), (iii), (iv), and (v), as described above). The final saliva dispersions with concentrations of 0, 2, 5, 10, 15 and 25 ng/mL of THC were mixed in a vortex.

After, 100 μL of the spiked samples with THC were added on the modified working electrode and then, the SWV was recovered under the same parameters explained in the section entitled “Sensor operation for THC detection in PBS buffer and simulated saliva”. Immediately after, the same sample previously tested with the modified working electrode was added on a bare working electrode (another portion of 100 μL). The samples prepared under the conditions from (i) to (iii) were tested with the modified working electrode by using 30 ng of THC initially deposited as was explained in the section entitled “Modification of the electrodes”. The samples prepared under the conditions (iv) and (v) were tested by using different amount of THC initially deposited on the working electrodes during the manufacturing of the modified working electrode. More specifically, an amount of 70, 85, and 100 ng of THC were initially deposited on the working electrode following the same procedure described in the section entitled “Modification of the electrodes”, with a slight variation like the water: methanol ratio (4:1).

Then, the calibration curves of THC in real saliva samples prepared under different conditions (from (i) to (v)), and using different modified working electrodes (30, 70, 85, and 100 ng of THC initially deposited) were performed. The values of the currents corresponding to each concentration result from subtracting the intensity of the peaks for the samples recovered with the modified working electrode (I_{mWE}) minus the signal obtained with the bare working electrode (I_{b}). The signals recovered by using the modified working electrode have the contributions of the interferences due to components of the saliva, the THCi deposited and the THC contained in the saliva. On the other hand, the signals obtained from the bare working electrode have the contribution of saliva components and the THC dispersed in the saliva. However, considering that this final contribution in the case of unmodified electrodes is almost negligible and indistinguishable from saliva, the signals recovered by using the bare working electrode is considered as “interferences”.

Validation and recovery analysis. The validation and recovery analysis was evaluated by using the following conditions: different saliva samples obtained from human subjects, treatment (iv), 100 ng of THC initially deposed on the modified sensing electrode, saliva: methanol (9:1), and concentration of THC spiked in the saliva samples of 0, 2, 5 and 10 ng/mL. This range of concentration corresponds to the results of the calibration curve. Then, the same procedure described in the section entitled “Sensor operation for THC detection in real saliva” was used to quantify the presence of THC in the samples and calculate the recovery percent. Besides, the sensor present herein was validated by testing the same samples from the different donors and spiked with different THC concentrations from 0 to 10 ng/mL and comparing with the concentrations quantified by ELISA THC Oral Fluid Kit from Neogen Corporation.

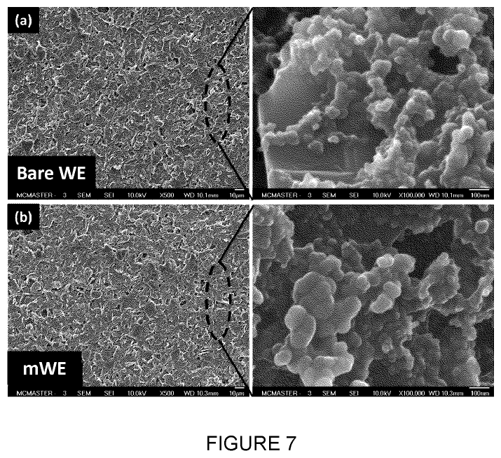

Characterization of the modified working electrodes. Carbon-based screen-printer electrodes purchased from Zensor R&D were used in this embodiment for the THC electrochemical sensor. The working electrode presents a geometric area of 0.07 cm² with an 85% effective electroactive area (0.06 cm²) according to the traditional determination by interrogating with cyclic voltammetric at different scan rates in ferricyanide/ferrocyanide solution. SEM micrographs (FIG. 7A) of the bare working electrode showed the typical scales-morphology due to graphite particles embedded within a binder polymeric material. In view of the traditional procedures to deal with screen printed electrodes, electrochemical pretreatment was considered and evaluated. Pretreatment electrochemical methods increased the electron transfer defining overlapped voltammetric peaks, and provided functional moieties to the electrode surface improving the absorption of analytes (data not shown). The bare working electrodes were interrogated with sulphuric acid, hydrogen peroxide, and a mix of both according to the best procedures reported in the literature to improve the electrode performance (González-Sánchez et al., 2019). However, despite an increase in the surface electroactive area and improved reversibility, the functional groups generated on the working electrode's surface interfered with the oxidation signal of THC by increasing the baseline.

Another option used to improve the electrochemical performance of the working electrodes was by chemical-mechanical polishing. The WEs were carefully polished with an adapted polishing pad and 3-μm aluminum till smooth mirror shine effect. The SEM micrographs showed the elimination of the polymeric residues on the electrode (data not shown), which improved the electrochemical performance during THC oxidation. However, for practical purposes in which a large amount of electrodes is required the reproducibility is a key parameter. The polishing of the electrodes resulted in improving but not reproducible electrochemical performance. Consequently, bare working electrodes just thoroughly washed with water were finally used despite the relatively reduced surface roughness leading a relatively slow electron transfer rates. However, considering that rougher surface settled by edge plane and defects sites are difficult to reproduce between electrodes, moderate roughness electrodes allow decreasing standard deviation of the sensor response.

As was explained in the section entitled “Modification of the electrodes”, 30 ng of THC contained in a stock solution of methanol: water (3:1) was dropped on the working electrode and interrogated in PBS (pH 7.4) by SVW. During this deposition, most of the molecules of THC (THCi) were oxidized and absorbed on the graphite material. As shown in the SEM micrographs of FIG. 7B, a non-variation on the morphology and roughness of the modified working electrodes was observed. On the other hand, the Raman spectrum showed slight variations between bare and the modified sensing electrodes (data not shown). For the bare working electrodes, the presence of two typical bands corresponding to D at ≈1337 cm⁻¹ and G at ≈1582 cm⁻¹ evidenced the graphitic carbon nature. The intensity ratio of both bands (ID/IG) around 1.7 suggested the poor crystallinity of the bare working electrode, with the possible presence of an amorphous phase (like carbon and inks components for example). The poor crystallinity may have affected the electrochemical performances of the electrodes. In the case of the modified working electrodes with the deposited of THCi, the D and G bands remained at the same wave number but a well-distinguished shoulder is visible at 1623 cm⁻¹. This peak is assigned in the literature due to the contribution of C═C stretch, C—H bend of benzene rings, and O—H bends from the THC molecules suggesting their deposition on the electrode.

Additionally, to the described deposition method above, other options were tested. For example, dropping the stock solution of THC directly in PBS (with different concentrations) and register the SVW after an incubation time; step by step deposition by dropping THC stock multiple times; recovery of different cycles of cyclic voltammetry of a solution of THC on the working electrode, and so on. Neither of the above deposition methods resulted in a homogeneous THC deposition between electrodes. Other optimization steps, like the electrochemical parameters required during the SVW, the amount of initial THC deposited on the working electrode, and the solvent nature of the stock solution were considered (data not shown).

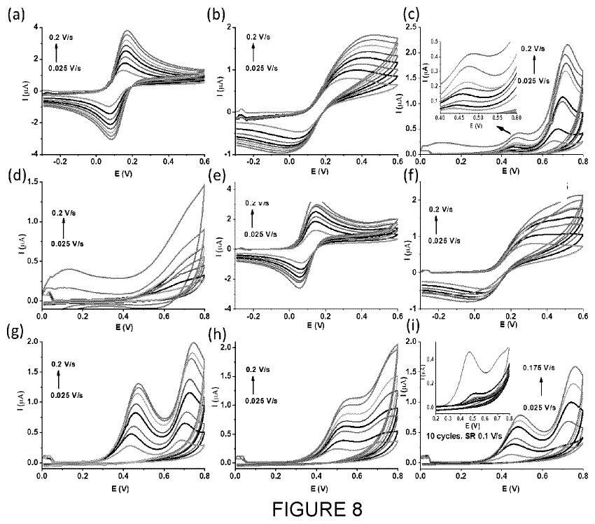

Electrochemical characterization. The electrochemical responses of a standard redox probe (K₄[FeCN₆] (FeCN) 0.1 mM in KCl 0.1 M), the bare working electrode and the modified working electrode in simulated saliva (SS), as well as THC (1000 ng/mL) dispersed in PBS pH 7.4 or SS were determined by interrogation with cyclic voltammetry (CV) at different scan rates (SR) (FIG. 8). The results shown in FIG. 8 provide information such as the influence of the viscosity of the media on the diffusion of the electroactive species, the reversibility (electron transfer), the absorption of the components of the SS on the bare working electrode and the modified working electrodes, the quantification of the number of THC molecules on the electrode's surface, the number of interchanged electrons, and the kinetic constant.

The reversibility performance of FeCN in KCl was not affected by using a modified working electrode (compare FIG. 8A with 8E). However, the reversibility performance was considerably affected in simulated saliva due to an increment of the viscosity of the media as well as the presence of a cocktail of components (compare FIG. 8A with 8B; 8E with 8F). Also, THC molecules in PBS showed an irreversible performance with an oxidation peak in the range of 0.45 to 0.5 V (FIG. 8C). However, in the presence of simulated saliva, the oxidation signal was lost probably due to the increment of the viscosity that affects the diffusion and the unwanted absorption of the different components of the SS on the electrode's surface.

When using the modified working electrode and PBS as the electrolyte, well-defined peaks were obtained in the range of 0.4-0.6 V, which corresponded to the oxidation of the molecules that remain adsorbed on the surface of the modified working electrode after the THC deposition. Also, after recording different cycles of the same modified working electrode, the signal was continuously decreasing but remained perceptible after 10 cycles. Also, once the THC is oxidized, some adduct species formed and might facilitate the further oxidation of other THC molecules. Higher intensities of the THC oxidation peaks were obtained by using the modified working electrode when compared to the bare working electrode in PBS (compare FIG. 8C with 8G) and most importantly in SS (compare FIG. 8D with 8H). In the modified working electrodes, the THC oxidation peaks correspond to the contribution to the deposited THCᵢ and the THC contained in the media (PBS or SS).

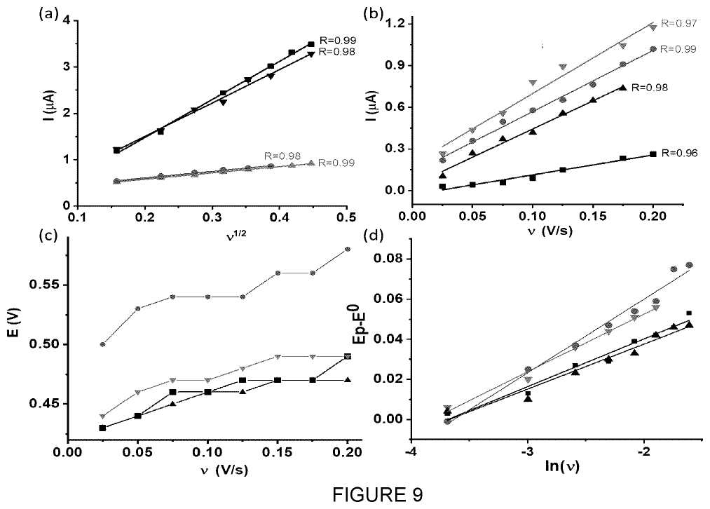

Different treatments of the CV data obtained by changing the SR allowed us to determine additional electrochemical performances and parameters of the different systems. According to the Randles-Sevcik equation, which describes how the peak current increases linearly with the square root of the scan rate (v^{1/2}), the FeCN in KCl and SS signal obtained in the bare and the modified working electrodes is attributed to an electrochemically reversible electron transfer processes involving freely diffusing redox species (FIG. 9A). The diffusion coefficients of FeCN in PBS and SS and by using the bare and the modified working electrodes were calculated through the Randles-Sevcik equation (Table 3). The diffusion coefficient of the FeCN species decreased by one order in the presence of simulated saliva due to higher viscosity. Also, the formal potential (E_{1/2}) of KCl increased in simulated saliva media due to the adsorption of components of the simulated saliva. It is reported that the effect of real saliva viscosity in electroactive species is even more noticeable than simulated media and calibration curves for sensors in real saliva should be performed instead of using analogs in simulated saliva.

The linear fit from the plot of the anodic current peak corresponding to the oxidation of THC and the scan rate (v) suggests that the oxidation of THC by using the bare and the modified working electrodes in PBS and SS is an adsorption controlled processes (FIG. 9B). Also, by increasing the scan rate, the oxidation peak of THC was shifted to a more positive potential (FIG. 9C). Considering the irreversible THC oxidation, the Laviron's equation and the plotting of Ep-E⁰ vs In(v) (in which Ep is the potential during the THC oxidation and E⁰ is the formal potential obtained from the intercept of the graphics of FIG. 9C) were used to estimate different parameters. As such, the surface concentration of THC on the electrode (Γ), the number of transferred electrons (n), and the reaction rate constants (ks) were calculated (Table 3). The Γ values suggested a higher amount of THC molecules adsorbed on the modified working electrode (when compared to the bare working electrode) as well as the negative effect of simulated saliva on the absorption of THC dispersed in SS on the modified working electrode. The number of electrons transferred during the oxidation is higher when THC is diluted in PBS when compared to SS. The modification of the electrodes improves the kinetics of the absorption-oxidation process of THC, and the SS ambushed the absorption-oxidation of the THC on the electrode. Therefore, it is expected that this effect will be remarkable in the case of real saliva.

TABLE 3
Electrochemical parameters.

| | Diffusion | | I′ | | |
|---|---|---|---|---|---|
| | coefficient | E_{1/2} | (mol/ | | Ks |
| | (cm²/s) | (V) | cm²) | n | (s⁻¹) |
| Bare working_FeCN_KCl | 2.3*10⁻⁵ | 0.12 | — | — | — |
| Bare working_FeCN_SS | 2.07*10⁻⁶ | 0.20 | — | — | — |
| Modified | 1.9*10⁻⁵ | 0.11 | — | — | — |
| working_FeCN_KCl | | | | | |
| Modified | 2.36*10⁻⁶ | 0.21 | — | — | — |
| working_FeCN_SS | | | | | |
| Bare working_THC_PBS | — | — | 6.3*10⁻¹¹ | 2.2 | 1711 |
| Modified working_PBS | — | — | 1.8*10⁻¹⁰ | 1.8 | 1609 |
| Modified | — | — | 2.2*10⁻¹⁰ | 2.4 | 2078 |
| working_THC_PBS | | | | | |
| Modified working_THC_SS | — | — | 1.9*10⁻¹⁰ | 1.4 | 1032 |

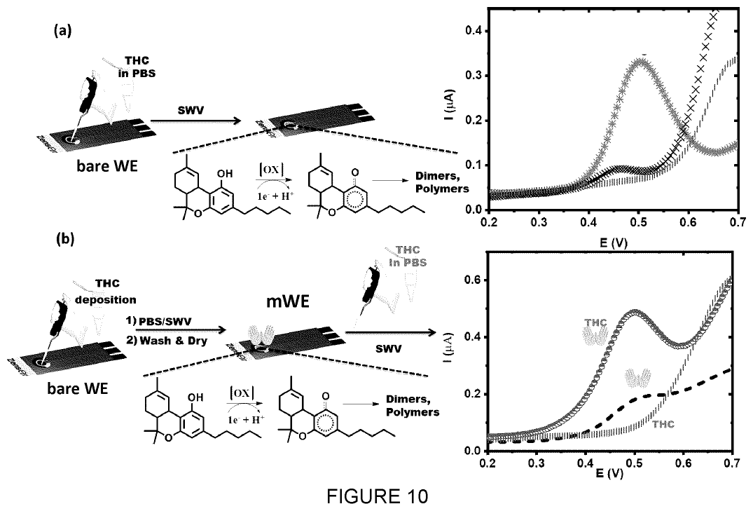

THC sensor performance in PBS and simulated saliva. The THC electrochemical sensor proposed herein is based on the oxidation of the hydroxyl group of the THC under an applied potential to form C═O moieties followed by the formation of adducts or more complex structures. In this sense, the bare working electrodes were used to interrogate different samples of THC in PBS via a voltammetric technique like square wave voltammetry (SWV) obtaining a well-defined oxidation peak between 0.4 to 0.5 V. However, the signals for low concentrations of THC (<100 ng/mL) were unidentifiable limiting the use of this approach for the detection of low levels of THC as is required in the market (FIG. 10A).

As was discussed before, during the THC record by SWV and using bare working electrodes, some molecules of the oxidized form or polymeric structures remain fixed on the electrodes through an adsorption controlled process. It was then tested if the deposited THC molecules (THCi) could be used as linker points to facilitate (“to lend a hand”) the interaction of the THC present in the samples with the electrode's surface. In such scenario, the signals corresponding to the deposited THC will be considered as the new “baseline or zero” (I₀) on which the final signals of the THC samples will be magnified. FIG. 10B compares the SWV signals of THC samples in PBS 10 ng/mL by using the bare and the modified working electrodes (with THCi).

Optimization of the conditions for the THC sensor performances. The THC sensor is based on the oxidation of the molecule under an applied potential by using a voltammetric technique and a modified working electrode. In this sense, different parameters were scrutinized such as the inputted parameters of the SWV technique (e.g., precondition potential and time, frequency, amplitude, etc); the effect of the ambient conditions (air, room temperature) on the modified working electrode features; the incubation time of the samples on the working electrode; the pH of the media; the different electrochemical voltammetric techniques, and so on.

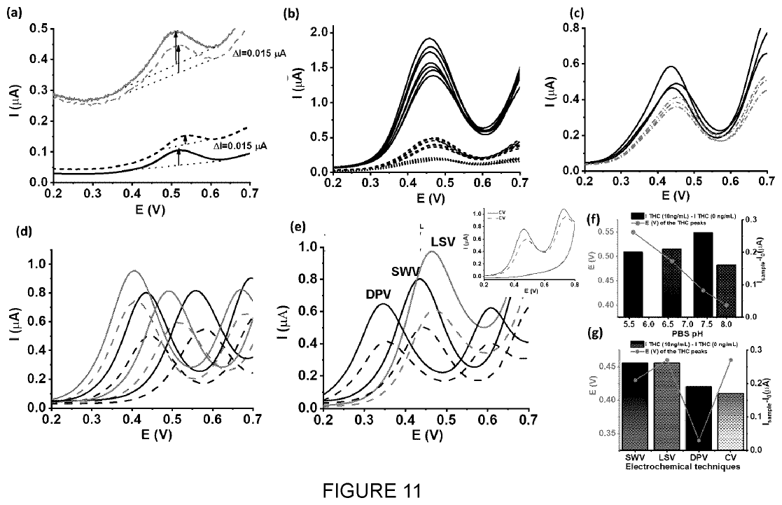

FIG. 11 shows some results during different optimizations. FIG. 11A compares the electrochemical performances of THC 10 ng/mL in PBS using the modified working electrode and two different SWV input parameters. In this sense, the conditions reported by Nissim and Compton (2015) to detect a low concentration of THC were compared with the parameters established in the present example (see section entitled “Sensor operation for THC detection in PBS buffer and simulated saliva”). In both cases, the same capability to detect THC 10 ng/mL was detected. The conditions reported herein presented lower SWV baselines allowing maintaining these parameters in the following experiences.

FIG. 11B shows the SWV of different electrodes during the deposition of THC (THCi) and the effect of ambient conditions like air and room temperature on the SWV of these modified working electrode in PBS (understood as the new zero), subsequently to the modification, and after 24 h. After 24 hours of manufacturing of the modified working electrode, the electrochemical feature of the layer of deposited THC on the electrode changed under ambient conditions consistent with continuous oxidation. For this reason, the modified working electrodes should be stored under an inert atmosphere (N₂) or vacuum until use. In this example, the modified working electrodes were freshly prepared before being used.

Different works in the literature report that increasing the incubation time of the samples containing the THC improves the adsorption of the molecules on the electrode's surface and hence the current intensity or the final analytical signal. However, as shown in FIG. 11C, the oxidation currents for samples of THC 10 ng/mL and using the modified working electrode decreased after 5 minutes of incubation time. This result is explained by the oxidation of THC on the surface of the modified working electrode and in the sample. For this reason, under the experimental conditions used, an incubation time higher than the precondition treatment time of 30 s seems ineffective.

The effects of pH on the SWV of samples of THC and the modified working electrode were studied. Increasing the pH of the PBS as the electrolyte media that contained the THC lowered the values of the potential oxidation (FIG. 11D). Also, at pH 7.4, the highest difference between the current of the sample of THC and the control or zero was obtained (FIG. 11F). According to this experiment, the samples were tested with a pH near 7.4. Finally, other voltammetric techniques (CV, cyclic voltammetry; LSV, linear sweep voltammetry; and DPV, differential pulse voltammetry) were evaluated and compared with SWV (FIG. 11E). Similar to the pH, the oxidation potential depends on the technique used. The lower overpotential with the best differentiation between the control and the sample THC 10 ng/mL corresponded to the SWV technique.

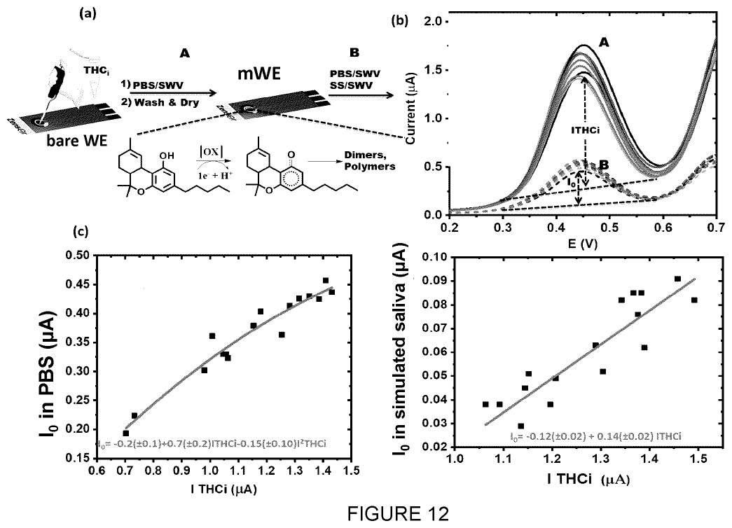

The reproducibility during the deposition of THCi was tracked by recovering the SWV peak for each electrode. Peaks with constant oxidation potentials and comparable currents were obtained in each batch for the optimized deposition (FIGS. 12A and 12B). However, even a small variation during the deposition affected the final sensor performance. For this reason, as quality control, samples of different batches of modified working electrodes were checked in PBS pH 7.4 and simulated saliva (FIGS. 12C and 12D). Also, the intensity of the signal (I₀) due to the oxidation of the THC previously deposited on the working electrode was registered. Then, I₀ was calculated for each modified working electrode with THCi (in PBS or simulated saliva) by using the showed mathematical A Zero (I₀) can thus be assigned to each electrode without the need for SWV interrogation in a blank solution before testing.

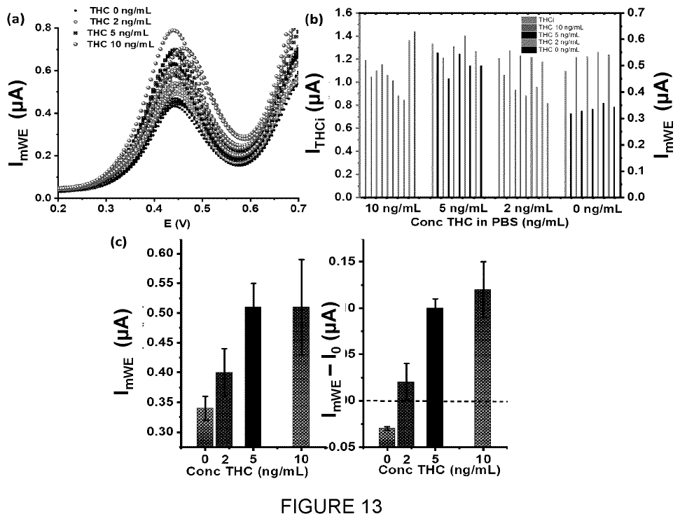

Calibration curve of THC in PBS and simulated saliva. The calibration curves were obtained by preparing samples of THC in PBS with different concentrations (0, 2, 5, 10, 100, and 1 000 ng/mL). Then, 100 μL were added to the modified working electrodes and interrogated with SWV. FIG. 13A shows an example of the SWV signals corresponding to each concentration (at least 5 individual modified sensing electrodes were used per sample). The current intensities were proportional to the concentration of THC but some variations corresponding to the same sample were obtained. As was explained before, slight variabilities during the initial deposition of THC result in different “zeros” (I₀) that could contribute to the deviation of the current intensities for the sample (FIG. 13B). For this reason, a correction was introduced. Instead of setting up the calibration curve by plotting the current intensities average corresponding to each concentration (FIG. 13C left), the I₀ (previously calculated as explained above) was subtracted from the current value tested by each electrode (FIG. 13C right). With this procedure, the differentiation between the signals regarding the concentrations was clarified.

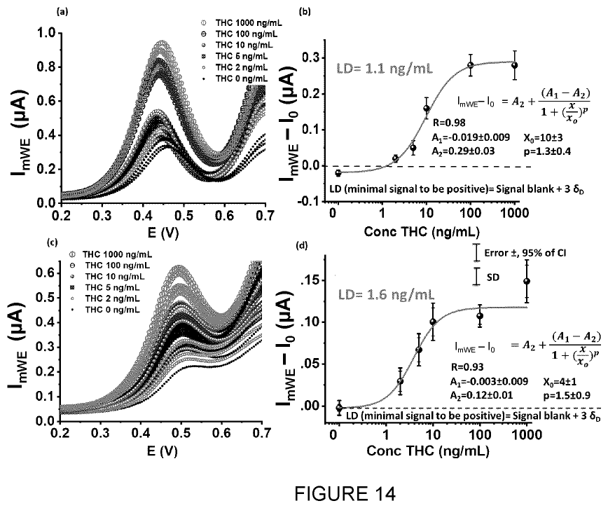

Following the previous procedure and including the corrections, the calibration curves for different concentrations of THC in PBS as well as in simulated saliva were recovered (FIG. 14). Both curves highly fit with a logistic function. In the case of PBS, the limit of detection was 1.1 ng/mL with a range of detection between 1.1 and 100 ng/mL (FIGS. 14A and 14B). In the case of simulated saliva, the current intensities for each concentration were lower than the analogs in PBS (FIG. 14C). This result is a consequence of the different components present in the simulated saliva (see table 2) which increase the viscosity that affects the diffusion, as well as unwanted absorptions on the electrode's surface (see section entitled “Electrochemical characterization”). Furthermore, the limit of detection was 1.6 ng/mL with a range of detection between 1.6 and 10 ng/mL (FIG. 14D).

Study of the interferences. Saliva is a viscous oral fluid based on water and a cocktail of many chemical species. Saliva samples vary in composition and properties between individuals and it is modulated by external stimuli. For studies in the lab, synthetic saliva was developed due to the use of natural saliva is limited, almost impossible to reproduce at different time, and difficult to collect and sterilize. In this example, simulated saliva (SS) was prepared by using components subdivided into three types: inorganic components (IC) made of inorganic ions; organic components (OC) formed by electroactive small organic molecules; and biological components (BC) based on a variety of proteins such as enzymes, mucus, and glycoproteins (Table 2).

For electrochemical sensors, different aspects of saliva composition should be taken into account. The viscosity of human saliva is around 1.30-times higher than water which therefore affects the diffusion of the analytes as well as the reaction rates on the electrodes (see section entitled “Electrochemical characterization”). The simulated saliva lacks the viscosity of authentic saliva which makes it difficult to extrapolate results. Furthermore, saliva components lead to a relatively stable pH normally ranged from 6.7 to 7.3, suitable values for this electrochemical approach (FIG. 10). In the present example, the interfering components were prepared in PBS pH=7.4 to control the pH as well as the ionic strength.

Finally, a variety of components of saliva might interfere with the electrochemical performance of the THC due to side effects like the adsorption process on the working electrode mainly by proteins and the presence of electroactive molecules. The simulated saliva used in this example (Table 2) was prepared by considering the high levels of three of the most important proteins present in the real saliva to mimick the effect of adsorption side effects. Since the THC detection is based on the oxidation signal around 0.4-0.5 V, different reported components of the saliva with oxidation reactions under potentials near 0.4 V were considered such as uric acid, glutamates, and ascorbic acid. The concentrations of such molecules were established based on the levels reported in the literature (Table 2).

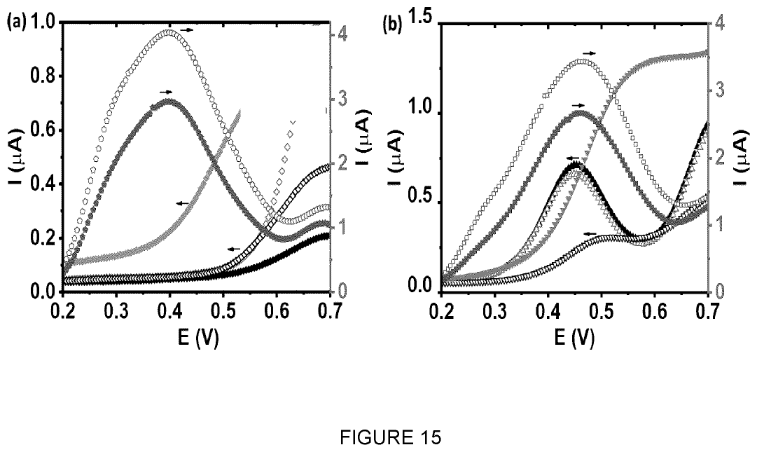

FIG. 15A shows the SWV response for the three types of interferents and using the bare working electrodes. For bare working electrodes, the IC and BC present a baseline similar to the PBS indicating no interference signals. Contrary, the OC presents a broad oxidation peak with a high current intensity whereby it is a considerable future interference during the THC detection. In this case, uric acid (UA) is present inside the OC with a concentration of 0.05 mg/mL, and is the main interference due to the oxidation near 0.4 V in PBS media. However, interesting is the fact that real saliva results in an electrochemical performance that differs from UA behavior. In this case, a shoulder with relatively high current intensities is obtained from 0.4 to 0.6 V. This rising current line signal corresponds to the effect of all the components of saliva including the UA. Fortunately, it is known that in real saliva the uric acid oxidation wave is shifted from that of simulated saliva to a more positive potential decreasing its effect as interference. This shift is impossible to control in synthetic saliva so the simulated saliva that was used during the THC calibration curve (see section entitled “Calibration curve of THC in PBS and simulated saliva”) does not contain uric acid.

FIG. 15B shows the SWV response for the three types of interferents and using the modified working electrodes. The signal corresponding to PBS is due to the deposited THC named as Zero and is consistent with the response of the inorganic component proving no interferences in this case. The BC solution affects the shape, intensity, and shifts the oxidation potential of the THC layer on the working electrode. This effect is due to the adsorption process as was explained before (see section entitled “Electrochemical characterization”) and it is also the cause of the decrement of the current obtained during the calibration curve of THC in simulated saliva comparing with PBS (see FIGS. 14B and 14D). Besides, the effect of the OC and UA is the same as for bare working electrodes, as well as, the real saliva showed a shoulder from 0.4 to 0.6 with associated higher current intensities than bare working electrodes. In this last case, the signal is different from the modified working electrode in PBS and is the result of the contributions of the saliva components and the THC deposited on the working electrode. This aspect lead to new modifications and optimizations of the approach to detect THC in real samples.

It is known that THC is metabolized to other species such as 11-OH-Δ⁹ tetrahydrocannabinol (11-15 OH-THC), 11-nor-9-carboxy-Δ⁹ - tetrahydrocannabinol (COON-THC), cannabinol (CBN), etc. The possible presence of such metabolites in saliva might affect the sensor performance if their oxidation potentials match with the THC, which is the principal psychoactive component of the cannabis. However, it is well known that the concentrations of 11-OH-THC, COOH-THC, and CBN in the saliva are considerably lower tan THC. Barely, frequent cannabis users might present residual COOH-THC content in saliva in contrast with occasional users.

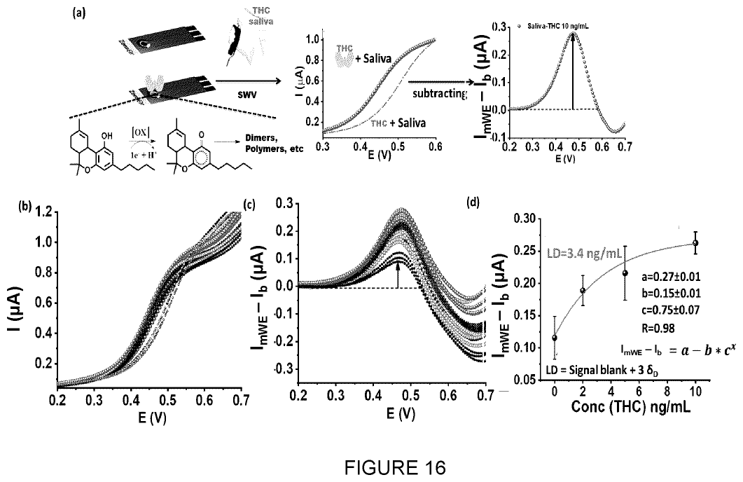

THC detection in real saliva samples. Considering the effect of real saliva on the electrochemical performance of the modified working electrode and THC oxidation peak, new calibration curves by using real saliva matrix were obtained. To accomplish the above, a variation to the sensor design was introduced. In this case, besides the detection of THC in saliva by using the modified working electrode and SWV as was explained before, immediately after, the same samples (containing different concentrations of THC) were interrogated with the bare working electrode under the same conditions (FIG. 16A).

The signals recovered by using the modified working electrode were modulated by the components of the saliva and the THCi deposited which also lend a hand to increase the absorption of the THC contained in the saliva. Furthermore, the signals obtained from the bare working electrode have the contribution of the saliva components and the THC dispersed in the saliva. However, the bare working electrode were not able to detect low concentrations of THC in saliva samples. Because of that, the SWV obtained with the bare working electrodes will be considered mainly as a result of the interferences present in the real saliva. Therefore, the removal of the interferences contributions to the analytical signal can be achieved by subtracting the data obtained from the modified working electrode minus the bare working electrodes. This approximation also allows working with different saliva samples no matter the composition or electrochemical performance. Finally, a well distinguishable peak was obtained with an oxidation current intensity proportional to the concentration of THC in the sample (FIG. 16A).

Different samples of saliva collected under the condition (i) (see section entitled “Sensor operation for THC detection in real saliva”) and spiked with THC with concentration from 0 to 10 ng/mL were evaluated by using the procedure described above and the modified working electrode obtained with 30 ng of THCi previously deposited (FIG. 16B). After the mentioned subtraction, well-defined peaks regarding the oxidation of THC were obtained with intensities proportional to the concentration of the analyte (FIG. 16C). The calibration curve with an exponential fit assurance a limit of detection of 3.4 ng/mL of THC in real saliva (FIG. 16D). However, the calibration curve lacked fair differentiation among the values corresponding to the different concentrations of 2, 5, and 10 ng/mL.

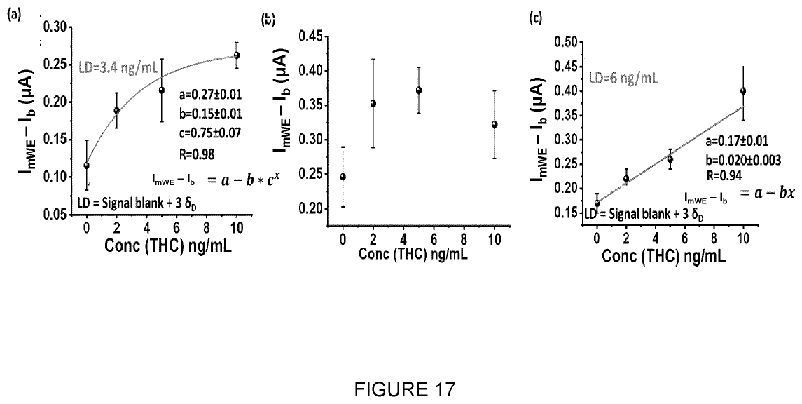

To deal with this inconvenience, some slight variations were proved like adjust the pH to 8 (see section entitled “Optimization of the conditions for the THC sensor performances”) (FIG. 17B) and dilution of the saliva with PBS ratio 1:1 (FIG. 17C). The change in the pH made the curve worse and the dilution improved the differentiation between 2, 5, and 10 ng/mL once the samples (4, 10, and 20 ng/mL) were diluted but increased the limit of detection to 6 ng/mL (FIG. 17D).

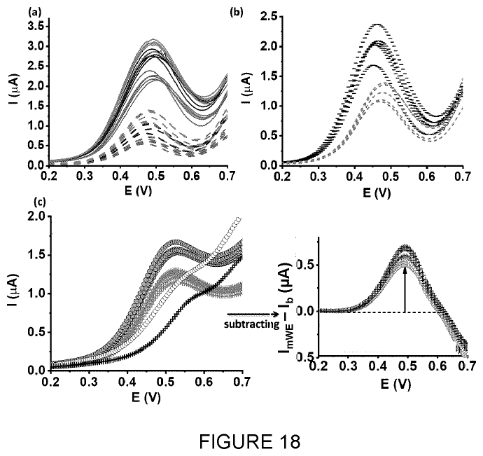

Considering the role of the THC immobilized on the modified working electrode to stimulate the absorption of the THC molecules containing in the sample with the working electrode and amplifying the final SWV signal, it was reasonable to assume that a higher amount of THC acting as the “hands” might improve the sensor performance. In this sense, different amounts (50, 70, and 100 ng) of THC initially deposited on the modified working electrode were evaluated. In this case, the higher amount of THCᵢ was deposited, the higher the current intensity of the signal corresponding to the “Zero” in PBS was obtained (FIG. 18A). In other words, the electrode's surface might increase the degree of linkers to interact with the THC in the sample benefiting the differentiation between the signals of the bare and the modified working electrodes during saliva testing.

Because methanol is a suitable solvent for the storage the THC and is used as the main component in some stabilizing buffers of THC samples, the effect of methanol on the electrochemical performance of THC using this approach was verified. FIG. 18B shows the increment of the SWV signals of the modified working electrode by adding to the PBS buffers a small portion of methanol (volume ratio PBS: methanol 9:1). For instance, the saliva matrix was diluted with methanol (volume ratio saliva: methanol 9:1) and the previous with PBS (volume ratio Saliva: methanol: PBS 5:1:4) and interrogated with bare and the modified working electrodes (FIG. 18C). After the corresponding subtraction, well-defined peaks were obtained due to the oxidation of the THCᵢ deposited with higher intensities than the analog without dilutions.

Calibration curves of samples of saliva containing different concentrations of THC were pursued by using the THCi amount deposition of 100 ng (other amounts of THCi such as 50 and 70 ng were tried with no success). Also, different dilution ratios corresponding to Saliva: methanol: PBS were evaluated (e.g., 900:100:0; 800:200:0; 800:100:100; 700:0:300; 700:50:300; 700:100:200; 700:200:100 and 500:100:400).

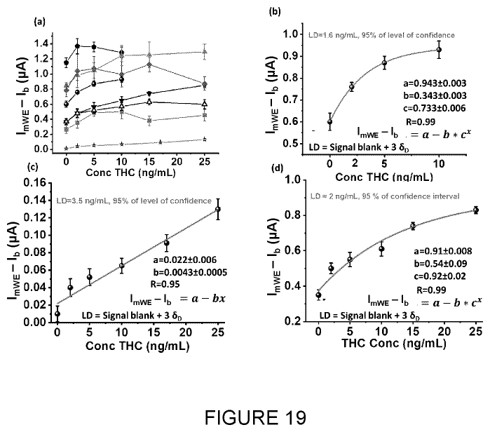

FIG. 19A shows the calibration curves of different samples of saliva spiked with THC and diluted with methanol and PBS (see section entitled “Electrochemical characterization”). The deviation of the value corresponding to each concentration is subject to many contributions like operator and instrument errors. However, an important issue is the reproducibility of the modified working electrode with THCi. In this case, after tracking 225 electrodes the current of THCi is around 2±0.2 μA, which is a 10% reproducibility affecting the final result. The higher amount of methanol, the higher the oxidation current of THC is observed in the deposited and the sample. However, the curves have a saturation zone around 5-10 ng/mL, being unable to be used for the detection of higher concentrations of THC. In this sense, PBS allows decreasing the saliva viscosity, fix the ionic strength of the media, and finally detect higher concentrations of THC.

In the case of Saliva: methanol (900:100) dilution, an impressive low limit of detection of 1.6 ng/mL was obtained as well as remarkable differentiation between concentrations of 0, 2, and 5 ng/mL (FIG. 19B). Concentrations higher than 5 ng/mL find on the flat portion of the curve, being impossible to quantify. It is important to point out the novelty of this result because so far the best sensor on the market for detecting THC in saliva presents a limit of detection of 5 ng/mL despite some laws and/or policies settled (in Canada and in the Untied States) of a limit of detection of THC as low as 2 ng/mL. On the other hand, Saliva: methanol: PBS (500,100,400) dilution of real samples spiked with THC concentrations of 0, 2, 5, 10, 17, and 25 ng/mL was effective to obtain a linear calibration curve with a LD of 3.5 ng/mL and well differentiated between the current values corresponding to a higher concentration than 10 ng/mL (FIG. 19C). In the case of dilution Saliva:PBS with no methanol (700:0:300), the limit of detection was 2 ng/mL being possible for the detection of a low level of the analyte as well as differentiation between the concentration of 2, 10, 15 and 25 ng/mL (FIG. 19D).

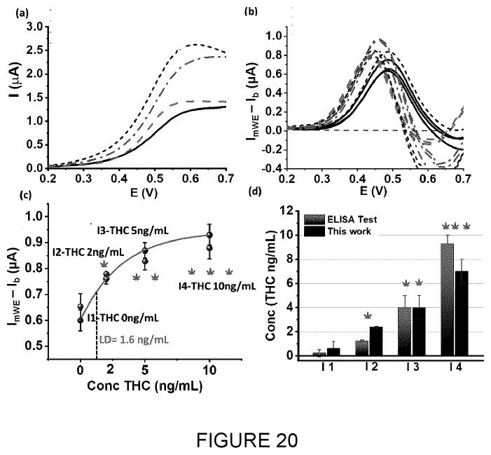

Validation and recovery of THC detection in real saliva. To corroborate the analytical accuracy of the electrochemical approach herein introduced, samples of saliva taken from 4 different healthy individuals spiked with low concentrations of THC were evaluated. First, FIG. 20A shows the remarkable differences in the electrochemical performance between the four individuals' saliva. This result confirms the expected assumption that each person presents a particular OF composition leads by several factors. The sensor presents herein considered the elimination of the interferences (by subtracting I from the modified working electrode the I from the bare working electrode signals, FIG. 20B). This design is useful to evaluate different saliva samples with the same calibration curve performed under similar matrix conditions.

FIG. 20C illustrates the interpolation in the calibration curve (FIG. 19B) of the values corresponding to each concentration of THC spiked in the individuals' saliva. Table 4 shows the calculated concentration of THC for each sample according to our approach and comparing it with ELISA test. The satisfactory recoveries of the THC ranged from 60% to 120% indicate that such a method has good specificity and applies to the quantification of THC in complex biological samples. The sample with THC 10 ng/mL presents the highest indetermination due to the flat portion of the curve in which it is contained. To quantify concentrations 5 ng/mL the calibration curve with other dilutions like Saliva:PBS dilution should be used.

TABLE 4
Validation and recovery percent.

| | Conc (THC ng/mL) | | | |
|---|---|---|---|---|
| | Added | Found | Found | |
| | according to | according to | according | Recovery |
| Individual | the standard | the ELISA | to this work | (%) |
| 1 | 0 | 0.2 ± 0.3 | 0.6 ± 0.6 (<LD 1.6 | — |
| | | | ng/mL) | |
| 2 | 2 | 1.3 ± 0.1 | 2.36 ± 0.06 | 115-121 |
| 3 | 5 | 4.6 ± 1.4 | 4.0 ± 1.0 | 60-100 |
| 4 | 10 | 9.3 ± 0.7 | 7.0 ± 1.0* | 60-80 |

*This value is not rigorously estimated.

Lastly, as a validation test, ELISA test was used to quantify the THC contained in the individuals' saliva samples. FIG. 20D depicts a comparison of the results obtained for both methods.

According to the hypothesis test of means of pair sample comparison, no significant difference between the data was observed at a confidence level of 95%. Therefore, the results showed suitable correspondence between the concentration values of THC obtained using the proposed electrochemical approach and those measured by the ELISA assay.

In this example, an original electrochemical sensor to detect low concentrations of THC in saliva was achieved by oxidation of such analyte through square wave voltammetry technique. To accomplish an ultra-low detection limit, THC molecules were initially deposited on the carbon-based working electrode assisting analyte-working electrode interaction and amplifying the final SWV signal. The saliva viscosity and composition disrupted the sensor performance controlled by absorption processes. Nevertheless, the effect of interferents present in the salive was handled by subtracting the signals of the interferences by testing the same sample with a bare working electrode. Thereby, the limits of detection were 1.1 ng/mL in PBS pH=7.4 media and 1.6 ng/mL in simulated and real saliva. Additionally, according to the dilution conditions the sensor was available to differentiate THC concentrations from 2 to 25 ng/mL in real saliva being competitive in market terms. Other attractive achievements were the duration of the test (38 seconds), the simple and profit-making manufacturing ability, drop-size sample volumes, portability, and hence the easy handling in field conditions.

### REFERENCES

González-Sánchez, M. I.; Gómez-Monedero, B.; Agrisuelas, J.; Iniesta, J.; Valero, E. Electrochemical Performance of Activated Screen Printed Carbon Electrodes for Hydrogen Peroxide and Phenol Derivatives Sensing. J. Electroanal. Chem. 2019, 839, 75-82.
Nissim, R.; Compton, R. G. Absorptive Stripping Voltammetry for Cannabis Detection. Chem. Cent. J. 2015, 9.

1. A sensor for detecting a sample analyte having an oxidizable phenolic group suspected to be present in a sample, the sensor comprising:
a sensing electrode;
a plurality of sensor analytes associated with the sensing electrode; and
a sample receiving region in fluid communication with the sensing electrode, wherein:
the plurality of sensor analytes facilitate, in the presence of an electric potential, (i) oxidation of the oxidizable phenolic group of the sample analyte to obtain an oxidized sample analyte, and (ii) association of a portion of the oxidized sample analyte with the sensing electrode; and
the plurality of sensor analytes comprise at least one of the following chemical species:
a first chemical species corresponding to the sample analyte having the oxidizable phenolic group;
a second chemical species corresponding to the sample analyte oxidized at the oxidizable phenolic group;
a third chemical species corresponding to a dimer, an oligomer or a polymer of the sample analyte oxidized at the oxidizable phenolic group; or
a combination thereof.

2. The sensor of claim 1, further comprising a baseline electrode, wherein the baseline electrode is a bare working electrode.

3. The sensor of claim 1, further comprising at least one substrate receiving the sensing electrode and/or the baseline electrode.

4. The sensor of claim 1, wherein the plurality of sensor analytes comprise a cannabinoid and/or is derived, at least in part, from oxidation, dimerization, oligomerization or polymerization of the cannabinoid.

5. The sensor of claim 4, wherein the cannabinoid is Δ⁹-tetrahydrocannabinol (THC).

6. The sensor of claim 1, wherein the sensing electrode comprises a carbon-based material, a nanomaterial, a metal-based material, or a combination thereof.

7. The sensor of claim 1, wherein the sample comprises a bodily fluid or a component of the bodily fluid.

8. The sensor of claim 7, wherein the sample comprises saliva or a component of saliva.

9. The sensor of claim 1 for detecting the sample analyte with a voltammetric technique.

10. The sensor of claim 9, wherein the voltammetric technique is square wave voltammetry, cyclic voltammetry, linear sweep voltammetry, or differential pulse voltammetry.

11. (canceled)

12. (canceled)

13. A process of fabricating a sensor for detecting a sample analyte having an oxidizable phenolic group suspected of being present in a sample, the process comprising:
(i) contacting a sensing electrode with a plurality of sensor analytes, wherein the plurality of sensor analytes comprises a first chemical species corresponding to the sample analyte having the oxidizable phenolic group; and
(ii) associating the plurality of the sensor analytes with the sensing electrode,
wherein:
the plurality of sensor analytes associated with the sensing electrode facilitate, in the presence of an electric potential, (i) oxidation of the oxidizable phenolic group of the sample analyte to obtain an oxidized sample analyte, and (ii) association of a portion of the oxidized sample analyte with the sensing electrode; and
the plurality of sensor analytes comprise at least one of the following chemical species:
the first chemical species corresponding to the sample analyte having the oxidizable phenolic group;
a second chemical species corresponding to the sample analyte oxidized at the oxidizable phenolic group;
a third chemical species corresponding to a dimer, an oligomer or a polymer of the sample analyte oxidized at the oxidizable phenolic group; or
a combination thereof.

14. The process of claim 13, wherein the plurality of sensor analytes are provided in a solution.

15. The process of claim 14, wherein the solution comprises an organic solvent.

16. The process of claim 15, wherein the organic solvent comprises methanol.

17. The process of claim 14, further comprising, prior to step (i), diluting the plurality of sensor analytes in an oxidizable form in the solution.

18. The process of claim 13, further comprising washing and/or polishing the sensing electrode.

19. The process of claim 13, wherein step (ii) comprises applying at least one potential to the sensing electrode to associate the plurality of the sensor analytes.

20. The process of claim 19, wherein step (ii) comprises electrodepositing the plurality of sensor analytes on the sensing electrode.

21. The process of claim 13, wherein the first chemical species of the plurality of sensor analytes is a cannabinoid.

22. (canceled)

23. The process of claim 13, further comprising, after step (ii), determining an electric current component resulting from the first chemical species of the plurality of sensor analytes associated with the sensing electrode in the presence of the electric potential.

24. (canceled)

25. A method of detecting a sample analyte having an oxidizable phenolic group suspected to be present in a sample, the method comprising:
receiving the sample on a sensor as defined in claim 1;
applying the electric potential to the sensing electrode to oxidize the sample analyte and obtain an oxidized sample analyte, and to associate a portion of the oxidized sample analyte with a surface of the sensing electrode;
obtaining a measured current from the sensing electrode while the at least one electric potential is applied;
comparing the measured current to a baseline current to determine a difference, the baseline current resulting from interfering electroactive agents in the sample; and
detecting a presence of the sample analyte when the difference exceeds a detection threshold.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)
